(19) United States
(12) Patent Application Publication
(10) Pub. No.: US 2026/0266836 A1
(43) Pub. Date: Sep. 10, 2026

(54) METHODS FOR DIAGNOSIS AND TREATMENT OF INFERTILITY

(71) Applicants: Thomas Jefferson University, Philadelphia, PA (US); Oregon Health & Science University, Portland, OR (US)

(72) Inventors: Dmitry Temiakov, Cherry Hill, NJ (US); William Lee, Philadelphia, PA (US); Shoukhrat Mitalipov, Beaverton, OR (US)

(21) Appl. No.: 19/152,205

(22) PCT Filed: Jan. 31, 2024
(86) PCT No.: PCT/US2024/013765

§ 371 Date: Jul. 30, 2025

Related U.S. Application Data
(60) Provisional application No. 63/442,622, filed on Feb. 1, 2023.

(51) Int. Cl. G01N 33/68; A61K 35/52; A61K 38/17

(52) U.S. Cl. CPC G01N 33/6872; A61K 35/52; A61K 38/1709; G01N 33/689; G01N 2800/367

(57) ABSTRACT

In one aspect, the present invention relates to a method of identifying an infertile or a hypofertile subject, wherein the method comprises comparing the sperm TFAM level from the spermatozoa of the subject with that of a reference TFAM level, wherein the lower level TF AM from the spermatozoa of the subject relative to the reference indicates that the subject is infertile or hypofertile.

### CROSS-REFERENCE TO RELATED APPLICATION(S)

This application claims the benefit of priority under 35 U.S.C. § 119 (e) to U.S. Provisional Patent Application No. 63/442,622, filed Feb. 1, 2023, the contents of which are incorporated by reference herein in their entirety.

### REFERENCE TO SEQUENCE LISTING

The Sequence Listing concurrently submitted herewith as a xml file named “205961-7096WO1 (00375)_Sequence Listing.xml” created on Jan. 30, 2024 and having a size of 144 Kilobytes is herein incorporated by reference pursuant to 37 C.F.R. § 1.52 (e) (5).

### BACKGROUND OF THE INVENTION

Infertility affects millions of people worldwide and has an impact on their families and communities. Currently, 15% of couples present difficulties to conceive and about 40%-50% of these cases are due to male infertility, which can result from low spermatozoa (sperm) production and various underlying factors affecting the quality of the produced sperm.

To evaluate the quality of semen, essential sperm parameters are evaluated including sperm concentration, motility, and morphology. Despite having apparently normal sperm parameters, the origin of male infertility remains unknown for 50% of individuals classified as having a normozoospermia. Additionally, although various Assisted Reproductive Technologies (ARTs) are being provided for achieving pregnancy for infertile males, the success of these technologies remains a challenge. Therefore, there exists a need to identify the exact causes of male infertility and accordingly develop methods for improving the success rate of ART for infertile males. The present invention addresses this unmet need.

### SUMMARY OF THE INVENTION

In one aspect, provided herein is a method of providing an assisted reproduction treatment to an infertile or a hypofertile subject, wherein the assisted reproduction treatment comprises supplementing the infertile or the hypofertile subject's spermatozoa with an effective amount of recombinant transcription factor A, mitochondrial (TFAM) during fertilization to improve its fertility.

In another aspect, provided herein is a method of identifying an infertile or a hypofertile subject, wherein the method comprises determining the expression level and/or the molecular weight of sperm TFAM in a spermatozoa sample prepared from semen obtained from the subject, and comparing the expression level and/or the molecular weight of sperm TFAM from the sample with the expression level and/or the size of sperm TFAM from a reference.

In yet another aspect, provided herein is a method of improving fertility of a spermatozoa of an infertile or a hypofertile subject, wherein the method comprises identifying the infertile or the hypofertile subject as described elsewhere herein and supplementing the infertile or the hypofertile subject's spermatozoa with an effective amount of recombinant TFAM during fertilization to improve its fertility.

In yet another aspect, provided herein is a method of diagnosing and treating an infertile or a hypofertile subject, wherein the method comprises identifying the infertile or the hypofertile subject as described elsewhere herein and providing assisted reproduction treatment to the subject identified as infertile or hypofertile, wherein the assisted reproduction treatment is as described elsewhere herein.

In yet another aspect, provided herein is a composition for improving fertility of spermatozoa of an infertile or a hypofertile subject, wherein the composition comprises a fertilization mix for Intracytoplasmic Sperm Injection (ICSI), wherein the fertilization mix includes oocyte(s) in Tyrode albumin lactate pyruvate medium (TH3) and an effective amount of recombinant TFAM, wherein the composition further includes spermatozoa for injecting into the oocyte(s).

In yet another aspect, provided herein is a kit comprising reagents for ICSI procedure, wherein the kit comprises Tyrode albumin lactate pyruvate medium (TH3), modified human tubal fluid, a recombinant TFAM solution, and an instructional material for use thereof.

In certain embodiments, the infertile or the hypofertile subject is the subject in whom the expression level and/or molecular weight of sperm TFAM is/are reduced relative to the expression level and/or the molecular weight of sperm TFAM from a reference, wherein the reference comprises spermatozoa from a fertile subject. In certain embodiments, the reference comprises spermatozoa from a group of fertile subjects. In certain embodiments, the reference comprises a predetermined value for expression level of sperm TFAM in a fertile subject or a group of fertile subjects.

In certain embodiments, the assisted reproduction treatment comprises Intracytoplasmic Sperm Injection (ICSI) treatment, wherein an effective amount of recombinant TFAM is used to supplement spermatozoa of the subject during fertilization.

In certain embodiments, the reference expresses about 300,000-500,000 TFAM molecules per spermatozoon. In certain embodiments, the reference expresses about 30 ng to about 100 ng of sperm TFAM per five million spermatozoa. In certain embodiments, the reference sperm TFAM has a molecular weight of about 30 kDa.

In certain embodiments, the supplementing comprises injecting the recombinant TFAM into an oocyte.

In certain embodiments, the expression level and/or the molecular weight of sperm TFAM is determined using at least one selected from the group consisting of Western blot, enzyme-linked immunosorbent assay (ELISA), mass spectroscopy, and confocal microscopy.

In certain embodiments, the subject is a mammalian male. In certain embodiments, the subject is a human male.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1E illustrate that mitochondria of human spermatozoa contains no mtDNA. FIG. 1A: schematic of the mtDNA copy number measurements in sperm cells by Droplet Digital PCR (ddPCR). FIGS. 1B-1C: representative 1D droplet scatter plots of ddPCR analysis of spermatozoa samples. The multiplex analysis of amplification of two single-copy nuclear genes, TATA-box binding protein1 and TEFM (FIG. 1B), and of two mitochondrial genes, cytochrome b, and ND1 (FIG. 1C) is presented. Dots above the thresholds (dotted lines) indicate droplets positive for the target amplicons. FIG. 1D: summary data showing mtDNA copy number per genome. FIG. 1E: in situ RNAScope labeling of mtDNA in developing sperm cells in seminiferous tubules of testicular tissue. mtDNA shown with arrows.

FIGS. 2A-2H illustrate that mature sperm cells contains transcription factor A, mitochondrial (TFAM) in the nucleus but not in the mitochondria. FIG. 2A: mature spermatozoa contain a specific isoform of TFAM. Western blot of mitochondrial lysate of HEK cells (lane 1), spermatozoa (lane 2), and spermatozoa in which the tails/midpieces have been removed (lane 3) are shown. Bottom-Western blot of the same gel stained with the anti-VDAC antibody. FIG. 2B: sperm isoform of TFAM is a product of alternative splicing of the TFAM pre-mRNA. Northern blot of mRNA extracted from sperm or HEK cells. FIG. 2C: schematic illustration of TFAM mRNA structure in somatic and sperm cells. PAS—polyadenylation signal. FIG. 2D: sperm TFAM isoform contains a mitochondrial targeting sequence (MTS) as revealed by LC-MS/MS data. FIGS. 2E, 2F: confocal microscopy shows the localization of the endogenous TFAM in mitochondria of HeLa cells (FIG. 2E) and the head of spermatozoa (FIG. 2F). Mitochondria staining with an anti-TOM20 antibody, and nucleus-DAPI. FIG. 2G: deconvolved 3D reconstructed confocal Z-stack image of spermatozoa. Staining with the antiacrosin antibody (left) or anti-acrosin and anti-TFAM antibody (right). FIG. 2H: cryo immunogold electron microscopy of spermatozoa cells reveals nuclear localization of TFAM. The head region is shown. Arrows indicate gold particles. A-acrosome, NC-nucleus.

FIGS. 3A-3D illustrate that MTS, but not UTRs of TFAM mRNA play role in its localization. FIG. 3A: UTR regions are not essential for TFAM localization in somatic cells. HeLa cells were transduced with the TFAM-mScarlet fusion having the nuclear 3′ and 5′ UTRs. FIG. 3B: only MTS is required for mitochondrial localization of TFAM in somatic cells. HeLa cells were transduced with TFAM-mScarlet fusion lacking the first 42 amino acids. FIGS. 3C, 3D: over-expressed TFAM-mScarlet fusion protein is found in the cytoplasm of sperm cells.

FIGS. 4A-4H illustrate that TFAM import to mitochondria is prevented in spermatozoa. FIG. 4A: MTSTFAM-mScarlet protein is localized to the sperm cytoplasm. FIG. 4B: MTSpol-D42TFAM-mScarlet is localized to the sperm mitochondria. FIG. 4C: S34 residue in the MTS of the sperm TFAM is phosphorylated. The MS/MS spectrum confirming the presence of Ser34 in TFAM is shown. m/z, mass-to-charge ratio. FIG. 4D: conservation of the serine residues implicated in TFAM phosphorylation in mammalian species. FIG. 4E. TFAMS31A/S34A-mScarlet protein is localized to the sperm mitochondria. FIG. 4F: schematic of a transversal cut of a seminiferous tubule. FIGS. 4G, 4H: human testicular tissue staining using DAPI (FIG. 4G) and an anti-TFAM antibody (FIG. 4H). arrows-spermatogonia, spermatocytes, spermatids. The basement membrane is indicated by a dashed line. L—seminiferous tubule lumen.

FIGS. 5A-5F illustrate that TFAM is released during nucleus decondensation. FIGS. 5A, 5B: TFAM remains in spermatozoa heads upon induced acrosome reaction. Sperm cells were stained with anti-TFAM and anti-acrosin antibodies. FIGS. 5C, 5D, 5E: TFAM is released during nucleus decondensation. Arrows indicate trace amounts of TFAM signal in the nucleus upon CTAB treatment (FIG. 5E). FIG. 5F: TFAM is imported into oocyte mitochondria upon microinjection. TD-oocyte image in transmission light. Oocyte staining with TFAM-mScarlet and anti-TOM20 antibodies.

FIGS. 6A-6B illustrate that sperm TFAM is a principal component of a quality control mechanism of fertilization. FIG. 6A: TFAM redistribution during sperm maturation. During sperm maturation, TFAM is phosphorylated and prevented from being imported to mitochondria, resulting in mtDNA degradation. Instead, TFAM is accumulated in the sperm nucleus. FIG. 6B: role of the paternal TFAM in fertilization. TFAM (dots) in the sperm nucleus enters the oocyte during fertilization (upper panel). TFAM is released into the cytoplasm upon nucleus decondensation (middle panel). Due to the presence of MTS, the paternal TFAM is destined for the oocyte mitochondria (low panel), where it can stimulate mitochondrial functions.

FIGS. 7A-7E illustrate mapping of the UTRs of the sperm TFAM isoform. FIG. 7A: Western Blot analysis of the proteins indicated in mitochondria of HEK cells and spermatozoa (SPZ). FIG. 7B: schematics of the RACE experiment. FIG. 7C: sequence of the 3′ UTR of the sperm TFAM cDNA. FIG. 7D: schematic illustration of polyadenylation of the TFAM mRNA in somatic cells. PAS-polyadenylation signal. FIG. 7E: schematic illustration of polyadenylation of the TFAM mRNA in sperm cells. Putative polyadenylation signal (PAS) and UGUAN elements are indicated.

FIGS. 8A-8C illustrate identification of TFAM peptides by LC-MS/MS analysis in HEK cells (FIG. 8A), human (FIG. 8B) and Rhesusmonkey (FIG. 8C) spermatozoa. Peptide search was done using MaxQuant. (The SGAELCSGCGSR (SEQ ID NO:52) peptide was identified in the human sperm TFAM sample using pFind and therefore not shown in panel B).

FIGS. 9A-9E illustrate TFAM localization in spermatozoa and somatic cells. FIG. 9A: cryo immunogold electron microscopy of spermatozoa head, arrows indicate gold particles. A-acrosome. FIG. 9B: cryo immunogold electron microscopy of spermatozoon midpiece. M-mitochondria. FIG. 9C. staining of human testicular tissue. Merge image. staining with anti-TFAM antibody, DAPI, staining with anti-TOM20 antibody, arrows-spermatogonia, spermatocytes, spermatids. The basement membrane is indicated by a dashed line, L—seminiferous tubule lumen. Close-up images of spermatogonia (1), spermatocytes (2) and spermatids (3) correspond to the squares indicated. FIG. 9D: over-expressed TFAM having the somatic 5′ and 3′ UTRs shows mitochondrial localization in HeLa cells. FIG. 9E: overexpressed TFAM lacking UTRs shows mitochondrial localization in Hela cells.

FIGS. 10A-10E illustrate expression of TFAM, H2B, TEFM, and TOM20 in somatic cells and mature spermatozoa. FIG. 10A: over-expression of TFAM mRNA having sperm 3′ and 5′ UTR regions result in cytoplasmic localization of this protein in spermatozoa. FIG. 10B: over-expression of H2B in HeLa cells shows nuclear localization. FIG. 10C: over-expression of H2B in sperm cells results in cytoplasmic localization. FIG. 10D: over-expression of TEFM shows mitochondrial localization in spermatozoa. FIG. 10E: over-expression of TOM20 results in mitochondrial localization in spermatozoa.

FIGS. 11A-11D illustrate trafficking of the TFAM variants in Hela cells. FIG. 11A: MTSₚₒₗ-mScarlet protein is localized to the sperm mitochondria. FIG. 11B: MTSₚₒₗ-mScarlet is localized to mitochondria in HeLa cells. FIG. 11C: MTS_{Po}l-D42TFAM is localized to the nucleus of HeLa cells. FIG. 11D: MTSTFAM-Scarlet is localized to mitochondria in HeLa cells.

### DETAILED DESCRIPTION

Uniparental inheritance of mtDNA is an evolutionary trait found in nearly all eukaryotes. Herein, it was identified that mitochondria in human spermatozoa are devoid of mtDNA and lack transcription factor A, mitochondrial (TFAM), the major nucleoid protein required to protect, maintain, and transcribe mtDNA, thereby explaining maternal inheritance of mtDNA. It was also found that the human spermatozoa contain an isoform of TFAM that retains the mitochondrial pre-sequence, which would ordinarily be removed during processing. Phosphorylation of this pre-sequence redirects TFAM to the spermatozoon nucleus, explaining the lack of mtDNA in mitochondria. During fertilization, paternal TFAM enters the oocyte cytoplasm, where it can be imported into maternal mitochondria to stimulate mtDNA replication and transcription. However, without wishing to be bound by any particular theory, it was contemplated that the presence of adequately expressed sperm isoform of TFAM is essential for successful fertilization.

The present disclosure, in one aspect, is related to identifying an infertile or a hypofertile subject (i.e., subject who lacks adequate amount and/or size of sperm TFAM) and providing the infertile subject with assisted reproduction treatment, wherein spermatozoa of the infertile subject are supplemented with a recombinant TFAM to enhance their fertility.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the term “antibody” refers to monoclonal antibodies (including full-length monoclonal antibodies), polyclonal antibodies, multispecific antibodies (e.g., bispecific antibodies), and antibody fragments. An antibody specifically binds to an antigen.

As used herein, the term “antigen” refers to a molecule to which an antibody can selectively bind. The target antigen may be a protein, carbohydrate, nucleic acid, lipid, hapten, or other naturally occurring or synthetic compound. Preferably, the target antigen is a protein or a complex of proteins.

As used herein, the term “biological sample” refers to a sample obtained from an individual and used in a diagnostic or monitoring assay. Biological samples encompass, e.g., a clinical sample, cells in culture, cell supernatants, cell lysates, plasma, serum, biological fluid (e.g., urine), and tissue samples. The source of the biological sample may be solid tissue (e.g., from a fresh, frozen, and/or preserved organ, tissue sample, biopsy, or aspirate), blood or any blood constituents, bodily fluids (such as, e.g., urine, lymph, cerebral spinal fluid, amniotic fluid, peritoneal fluid, or interstitial fluid, semen), or cells from any time in gestation or development of the individual. The biological sample may contain compounds that are not naturally intermixed with the tissue in nature, such as preservatives, anticoagulants, buffers, fixatives, nutrients, or antibiotics.

As used herein, the terms “biomarker” or “marker” generally refers to a nucleic acid molecule, clinical indicator, protein, or other analyte that is associated with a disease. In various embodiments, a biomarker is differentially present in a biological sample obtained from a subject having or at risk of developing a disease or disorder (for e.g., infertility) relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease, for determining the stage or type of a disease (e.g., acute disease or chronic disease), for determining the prognosis of the subject, for evaluating therapeutic efficacy, or for selecting a treatment regimen.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “disease” refers to a state of health of an animal wherein the animal cannot maintain homeostasis, and wherein if the disease is not ameliorated then the animal's health continues to deteriorate. A “disease subtype” is a state of health of an animal wherein animals with the disease manifest different clinical features or symptoms.

A “disorder” as used herein, is used interchangeably with “condition,” and refers to a state of health in an animal, wherein the animal is able to maintain homeostasis, but in which the animal's state of health is less favorable than it would be in the absence of the disorder. Left untreated, a disorder does not necessarily cause a further decrease in the animal's state of health.

As used herein, the term “effective amount” refers to the amount required to reduce or improve at least one symptom of a disease or disorder relative to an untreated patient or to practice the methods of the invention with a successful or enhanced outcome. The effective amount of active compound(s) used to practice the present invention for varies depending upon the manner of administration, the age, body weight, and general health of the subject as well as requirements of the methods of the invention.

As used herein, the term “endogenous” refers to any material from or produced inside an organism, cell, tissue or system.

As used herein, the term “fertilization” refers to the fusion of male and female gametes that facilitates the development of a new organism.

As used herein, the term “fertilization mix” refers to a mixture that comprises components for facilitating a successful fertilization.

As used herein “infertility” or “hypofertility” refers to a disease of the male or female reproductive system defined by the failure to achieve a pregnancy after 12 months or more of regular unprotected sexual intercourse, wherein the male or the female are deemed to be infertile or hypofertile. Infertility can be primary or secondary. Primary infertility is when a pregnancy has never been achieved by a person, and secondary infertility is when at least one prior pregnancy has been achieved.

As used herein “instructional material” includes a publication, a recording, a diagram, or any other medium of expression that can be used to communicate the usefulness of the device in a kit. The instructional material of the kit may, for example, be affixed to a container that contains the device of the invention or be shipped together with a container that contains the device. Alternatively, the instructional material may be shipped separately from the container with the intention that the recipient uses the instructional material and the device cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein the term “level” is intended to refer to amount of a unit of a compound being measured, for example a protein. It is also intended to encompass “concentration” expressed as amount per volume or weight per volume and any other depiction of concentration as known in the art.

As used herein the terms “patient,” “subject,” “individual,” and the like are used interchangeably herein, and refer to a human, or cells thereof whether in vitro or in situ, amenable to the methods described herein.

As used herein the term “pregnant” refers to the fertilization and development of offspring (embryo or fetus) in a women's uterus.

As used herein, the terms “prevent,” “preventing,” “prevention,” and the like refer to reducing the probability of developing a disease or condition in a subject, who does not have, but is at risk of or susceptible to developing a disease or condition.

As used herein the terms “purified” or “biologically pure” refer to material that is free to varying degrees from components which normally accompany it as found in its native state. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein the term “reference” is meant a standard or control value often used as a basis for comparison. The terms “reference” and “control” are used interchangeably herein. A “reference” or “control” means a value or level measured in a sample obtained from a fertile subject. A “reference” or “control” also means a value or level that is a composite of values or levels obtained from a group of subjects as described herein, or as generated by an algorithm using multiple measured values or levels.

The “size” of a protein refers to the number of amino acids it contains and/or its total molecular mass, which is normally reported in units of Daltons or kilodaltons (kDa).

As used herein, the term “subject” refers to a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

As used herein the term “immobilized” refers to bound directly or indirectly to a surface of, e.g., a device, including attachment by covalent binding or noncovalent binding (e.g., hydrogen bonding, ionic interactions, van der Waals forces, or hydrophobic interactions).

As used herein the term “signal” refers to light intensity (e.g., light generated by fluorescence, bioluminescence, or phosphorescence), ionizing radiation, particle emission, magnetism, staining, or a product of a reaction involving an enzyme. Diffraction, absorbance, polarization, reflection, deflection, increases, decreases, or amplification of a signal may be indicative of an event (e.g., binding of a biomarker or biomarker complex to an antibody immobilized on the surface of a diffraction-based device).

As used herein the term “specifically binds” refers to an antibody or fragment thereof that recognizes and binds an antigen, but that does not substantially recognize or bind to other molecules in a biological sample. Specific recognition of an antigen by an antibody may be assayed by using, e.g., light diffraction devices with an immobilized capture surface or using standard techniques known to one of skill in the art, such as immunoprecipitation, Western blotting, and ELISA.

As used herein the term “therapeutic” refers to a treatment and/or prophylaxis. A therapeutic effect is obtained by suppression, remission, or eradication of a disease state.

As used herein, the terms “treat,” treating,” “treatment,” and the like refer to reducing or improving a disease or condition and/or symptom associated therewith. It will be appreciated that, although not precluded, treating a disease or condition does not require that the disease, condition or symptoms associated therewith be completely ameliorated or eliminated. Additionally, these terms also refer to methods/procedures such as, for example, assisted reproduction provided to the subjects in need thereof.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

### Methods:

In one aspect, the disclosed herein is a method of providing assisted reproduction treatment to an infertile or a hypofertile subject. The method comprises identifying the infertile or the hypofertile subject and then accordingly providing the assisted reproduction treatment to the infertile or the hypofertile subject. In certain embodiments, the assisted reproduction treatment includes Intracytoplasmic Sperm Injection (ICSI) treatment. In certain embodiments, the assisted reproduction treatment is ICSI treatment.

In certain embodiments, identifying the infertile or the hypofertile subject comprises the steps of determining and comparing the expression level and/or the molecular weight of sperm TFAM from a spermatozoa sample of the subject with the expression level and/or the molecular weight of sperm TFAM from a reference, wherein a lower expression level and/or reduced molecular weight of sperm TFAM from the sample relative to the expression level and/or the molecular weight of sperm TFAM from the reference indicates that the subject is infertile or hypofertile.

In certain embodiment, the decrease in the expression level and/or the molecular weight of sperm TFAM from the sample compared to that of the reference can be as little as about 1%, 2%, 3%, 4%, 5%, 10%, 20%, 30%, or about 40%, 50%, 60%, or even by as much as about 70%, 75%, 80%, 90%, or 100%.

In certain embodiment, the reference is spermatozoa of a fertile subject. In certain embodiments, the reference is spermatozoa of a group of fertile subjects. In certain embodiments, the reference comprises a predetermined value of the expression level of sperm TFAM from a fertile subject or a group of fertile subjects.

In certain embodiments, the molecular weight of the sperm TFAM from the reference is about 30 kDa. In certain embodiment, the reference comprises about 300,000-500,000 molecules of sperm TFAM per a sperm cell. In certain embodiments, the expression level of sperm TFAM in the reference amounts to about 30 to about 100 ng sperm TFAM per 5 million cells.

In certain embodiments, the expression level and/or the molecular weight of the sperm TFAM can be determined using, for example, Western Blot analysis with sperm TFAM-specific antibodies, ELISA with sperm TFAM-specific antibodies, mass spectroscopy or any combination thereof.

In certain embodiments, the expression level and/or the molecular weight of sperm TFAM are determined, for example, using Western Blot analysis. As an example, Western Blot analysis comprises the steps of: preparing spermatozoa sample from semen obtained from the subject by, for example, by using gradient centrifugation; lysing the spermatozoa sample, for example, by sonication; separating the protein in the sonicated samples on SDS-PAGE gels; blotting the separated proteins on a polyvinylidene difluoride membrane; blocking the membrane; incubating the membrane overnight with mouse monoclonal anti-TFAM antibody; washing the membrane; incubating the membrane with IRDye 680RD-labeled goat anti-mouse antibody; detecting fluorescent signal using Infrared Imaging System.

In certain embodiments, ICSI (Intracytoplasmic Sperm Injection) is used as an assisted reproduction technology (ART) for providing assisted reproduction to the infertile or the hypofertile subject. ICSI is an in vitro fertilization (IVF) procedure in which a single sperm cell is injected directly into the cytoplasm of an egg. This technique is used to prepare the gametes for the obtention of embryos that may be transferred to a uterus. Using this method, the acrosome reaction is skipped.

In certain embodiments, the Intracytoplasmic Sperm Injection (ICSI) treatment used herein includes the steps of: mounting the dish comprising oocytes and an effective amount of recombinant TFAM (rTFAM) on the stage of an inverted microscope equipped with a stage warmer and micromanipulators, immobilizing the spermatozoa on the lid of the dish with a microinjection pipette; aspirating the spermatozoa and rTFAM into an ICSI pipette and injecting it into the oocyte cytoplasm, away from the polar body. The injected oocytes are cultured in a suitable medium. Fertilization is determined approximately after ICSI by noting the presence of two pronuclei and second polar body extrusion in the oocyte.

In another aspect, disclosed herein is a method of improving fertility of spermatozoa from an infertile or a hypofertile subject. The method comprises identifying the infertile or the hypofertile subject, wherein the method of identifying the infertile or the hypofertile subject is as described elsewhere herein. Supplementing the spermatozoa of the identified infertile or hypofertile subject with an effective amount of a rTFAM during the ICSI treatment to improve the fertility of the spermatozoa of the infertile or the hypofertile subject.

In certain embodiments, rTFAM is a human recombinant TFAM having modification(s) to improve its import to oocyte mitochondria. In certain embodiments, the subject is a mammalian male. In certain embodiments, the subject is a human male.

### Composition:

In another aspect, disclosed herein is a composition for improving fertility of spermatozoa of an infertile or a hypofertile subject. The composition comprises a fertilization mix suitable for assisted reproduction technique such as, for example, ICSI; wherein the fertilization mix includes oocytes in Tyrode albumin lactate pyruvate medium (TH3) and an effective amount of rTFAM, wherein the composition further includes spermatozoa for injecting into the oocytes.

In certain embodiment, the rTFAM is produced in E. coli cells and purified using affinity chromatography. In certain embodiment, the rTFAM is produced in human cells and purified using affinity chromatography. In certain embodiment, the rTFAM is further modified to improve its solubility and import into mitochondria. In certain embodiments, the purified rTFAM is dissolved a suitable buffer, such as PBS buffer, sterilized, and stored at suitable lower temperatures or using with the ICIS treatment, as described elsewhere herein.

In certain embodiments, the rTFAM is as described elsewhere herein.

### Kit:

In yet another aspect, the invention provides a kit comprising reagents for ICSI, wherein the kit comprises Tyrode albumin lactate pyruvate medium (TH3), a modified human tubal fluid, HEPES, a rTFAM solution and an instruction material for the use thereof. In certain embodiments, the rTFAM is as described elsewhere herein.

It should be understood that the method, the compositions and the kits that would be useful in the present invention are not limited to the particular formulations set forth in the examples. The following examples are put forth so as to provide those of ordinary skill in the art with a complete disclosure and description of how to make and use the cells, expansion and culture methods, protein purification methods, and therapeutic methods of the invention, and are not intended to limit the scope of what the inventor regards as his invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

The materials and methods employed in the experiments disclosed herein are now described.

### Materials and Methods

### Cell Culture

HEK293, HEK293T, and HeLa cells were cultured in DMEM containing 10% (v/v) FBS and 1% (v/v) Penicillin/Streptomycin (all from Gibco) at 37° C. and 5% CO₂ in a humidified atmosphere. Adherent cells were harvested by incubation with 0.25% trypsin-EDTA. Cells were passaged and maintained at a 2.5×10⁵ cells/ml density.

### Gamete Collection and Analysis.

Semen collection was obtained via at-home semen collection kits or at a Fertility Clinic. Participants were instructed to abstain from ejaculation for 24 h prior to collection. The ejaculate was maintained at body temperature until processing. Sperm samples were obtained from normozoospermic fertile adult donors (34-43 yo). The samples were liquefied for 30-60 min at room temperature (RT). After liquefaction, the semen was loaded on AllGrad 90% (Cooper Surgical, #AG90-100) gradient solution and centrifuged at 1500 rpm for 15 min. The pellet containing spermatozoa was then rinsed in Quinn's sperm washing medium (Cooper Surgical, #ART-1006) and centrifuged again at 1100 rpm for 6 min. The cells were counted using a hemocytometer and observed for the presence of the contaminating cells. Sperm from the adult Rhesus macaque (Macaca mulatta) was collected as described previously.

Measurement of the Absolute Copy Number of mtDNA

Purified human sperm cells (4,000,000 cells in 2 μl) were mixed with 8 μl of the pre-warmed lysis solution containing 10 M Urea and 30 mM DTT and incubated for 5 min at 95° C. Upon incubation, the lysed cells (5 μl) were transferred into a 1.5 ml tube containing 1 ml of DNA/RNA/protein solubilization reagent 100ST (DireCtQuant), mixed by vortexing, and incubated for 10 min at 95° C. to complete the lysis and to dilute the sample to the optimal concentration of Urea (less than 50 mM). The resulting sample containing 2,000 lysed spermatozoa cells per μl of solution was used to measure the absolute copy number of mtDNA by Droplet Digital PCR (ddPCR). MtDNA was amplified using two primer pairs, mt64-ND1, and mt92-CYTB, which target two opposite regions of the mtDNA genome, as described.

The droplet reaction consisted of 1× QX200™ ddPCR™ EvaGreen Supermix (1864033, Bio-Rad), a 0.1 μl 1 aliquot of the target sample, and TBP73 (95 nM) and TEFM88 (140 nM) primers for genome analysis, or mt64-ND1 (100 nM) and mt92-CYTB (135 nM) primers for mtDNA analyses. A restriction enzyme digestion was performed prior to partitioning into droplets by adding the Fast Digest enzymes HaelII and MseI (0.5 U each) into the ddPCR reaction for nuclear genome, and AluI (1 U) for mtDNA amplification. The reactions were incubated for 15 min at 37° C. Non-template controls were included in each analysis plate to monitor possible reaction contamination. The PCR amplification was performed using the C1000 Touch Thermal Cycler (Bio-Rad) and the following thermal profile: 95° C. 5 min; (95° C. 30 sec; 60° C. 1 min) 40 repeats; 4° C. 5 min; 90° C. 10 min.

The data analysis was performed with QuantaSoft Analysis Pro v1.0 using thresholds to distinguish single and double-positive droplet populations. The number of genomes was calculated by averaging the DNA copy number obtained using TBP73 and TEFM88. To determine the mtDNA amount per haploid genome in a sperm cell, the number of mtDNA copies obtained with the mt64-ND1 or the mt92-CYTB amplicon was divided by the number of genomes measured in the same sperm sample. Simple or multiplex analyses were performed using the QX200 Digital Droplet PCR platform (Bio-Rad Laboratories).

### Chromogenic In Situ Hybridization Assay (CISH) Using Human Testicular Tissue

The study involving testicular tissues was conducted as approved. Human testicular tissues from three patients with testicular cancer were obtained. The patients were 28-29 yo and had no history of mitochondrial disease. Non-neoplastic human testicular parenchyma from the orchiectomy specimens was obtained. The tissues were sectioned at 4 μm in thickness, away from the tumor, and mounted onto slides. The tissues were fixed with 10% formalin for 24 h, immersed in a series of various concentrations of ethanol (70, 80, 95, 100%) and xylene followed by embedding into paraffin blocks with paraffin wax. Hematoxylin and eosin (H&E) staining has been performed. The H&E slides of testicular tissue were reviewed by a pathologist. The sections with normal spermatogenesis were marked, and adequate unstained sections were obtained from the formalin-fixed paraffin-embedded (FFPE) blocks for CISH and IHC.

CISH with mtDNA in testicular tissues was performed exactly as described in the manufacturer's protocol (Advanced Cell Diagnostic), using RNAscope 2.5 HD Assay BROWN Detection Kit and Hs-MT-COX1-sense probe (#478051; ACD). The signal was developed by incubation with DAB (3,3′-diaminobenzidine) for 10 minutes at RT and counterstained with hematoxylin (Sigma), followed by a wash in 0.02% ammonia water before mounting. Digital Images were obtained using Olympus BX60 microscope at 20× magnification and the cellSens imaging software (Olympus).

### Mitochondria Isolation

HeLa cells were gently homogenized with 25 strokes using a Teflon homogenizer (Thomas Scientifics) in 7.5 ml of HES buffer (20 mM HEPES, pH7.2, 0.25 M sucrose, and 0.1 mM EDTA), supplemented with 1 mM DTT, 0.1 mg/ml BSA, and 0.1 mM PMSF. Homogenates were centrifuged at 2,000 g for 5 minutes at 4° C. Supernatants were collected and centrifuged again at 10,000 g for 12 minutes. The crude mitochondria were resuspended in HES buffer and further purified by a sucrose step gradient separation (15/23/34/60% sucrose in 20 mM HEPES, pH 7.2, 0.1 mM EDTA) at 100,000 g at 4° C. for 1 h. The purified mitochondria were resuspended in 100 μl of HES buffer, aliquoted, and stored at −20° C.

### RNA Purification

To extract the RNA from human spermatozoa (~80 million cells in 100 μl), they were homogenized by vortexing with 100 mg of 0.2 mm stainless steel beads (Next Advance) in Lysis Buffer (Thermo Scientific), supplemented with guanidine thiocyanate and 2% 2-mercaptoethanol, for 5 minutes at RT. The RNA was isolated from the lysate using the GeneJET RNA Purification Kit (Thermo Scientific), aliquoted, and stored at −80° C. To extract the RNA from HEK293 cells, they were lysed in 1 ml of TRI Reagent (Invitrogen) for 5 minutes at RT. 1-Bromo-3-chloropropane (100 μl) was added, vortexed for 15 seconds, and incubated for 10 minutes at RT. The lysate was then centrifuged at 12,000 g for 15 minutes at 4° C. to separate the RNA-containing aqueous phase. The RNA was precipitated by mixing the aqueous phase with isopropanol and centrifuged at 12,000 g for 15 minutes at 4° C. The RNA pellet was washed with 75% ethanol and air-dried before resuspending in DEPC-treated water. The purified RNA was aliquoted and stored at −80° C.

### Mapping of the 3′ UTR Region of the Mitochondrial and Sperm Isoform of TEAM

RNA samples were first treated with dsDNAse (Thermo Fisher Scientific) to remove any DNA contamination. The 3′ ends of the sperm and HEK293 TFAM mRNA species were mapped by 3′ RLM-RACE using the FirstChoice RLM-RACE Kit (Ambion) with modifications. The RNA adaptor provided by the kit was phosphorylated on its 5′ end using T4 Polynucleotide Kinase (NEB). RNA (1 μg) was ligated to the 5′-phosphorylated RNA adaptor using T4 RNA Ligase (Ambion). Following ligation, the TFAM mRNA was amplified by RT-PCR using the SuperScript IV One-Step RT-PCR System (Thermo Fisher Scientific). The RT-PCR reaction was further amplified by PCR using inner primers (Shown in Table 2).

### LC-MS/MS Analysis

To reduce the complexity of the sample for LC-MS/MS analysis, the band representing sperm TFAM was excised from 10% PAGE. Spermatozoa cells (~5 million) were lysed in SDS sample buffer by sonication. Recombinant human TFAM was used as a protein marker to excise the band representing the sperm TFAM isoform. The excised protein band was washed and subjected to trypsin digestion. The gel bands were treated with tris(2-carboxyethyl) phosphine, alkylated with iodoacetamide, and the protein digested with trypsin. Tryptic digests were analyzed by LC-MS/MS using a Q Exactive Plus or Q Exactive HF mass spectrometer (ThermoFisher Scientific) coupled with a Nano-ACQUITY UPLC system (Waters) as previously described. Peptides and proteins were identified using MaxQuant. MS/MS spectra were searched against the UniProt human or rhesus macaque protein database and a common contaminant database using full tryptic specificity with up to two missed cleavages and static carbamidomethylation of Cys. Variable modifications included in the search were oxidation of Met, deamidation of Asn, and protein N-terminal acetylation. To identify the TFAM maturation site in HEK mitochondria, a semi-tryptic search was performed. For identification of unknown PTMs, the data was analyzed using the open search function of pFind 3.1.5 with a precursor and fragment mass tolerance of 10 ppm. Consensus identification lists were generated with false discovery rates set at 1% for protein, peptide, and site identifications.

### Northern Blotting

The sperm and HEK293 RNA samples were diluted in a loading buffer (95% formamide, 5 mM EDTA, 0.025% SDS, 0.025% bromophenol blue, and 0.025% xylene cyanol) and heated at 95° C. for 3 minutes to further denature samples before loading. RNA (1 μg) was loaded onto a 6% PAGE containing 6M Urea in 1× Tris-Borate-EDTA (TBE) buffer. HEK293 RNA (50 ng) was loaded onto the gel, and detection of 18S rRNA was used as a size marker. The RNA species were transferred onto a Hybond-H+ membrane (GE Healthcare) in 1× TBE buffer and crosslinked by UV exposure for 2 minutes upon electrophoresis. The DNA probes (Table 2) at 500 nM were 5′-[³²P]-labeled using T4 Polynucleotide Kinase (NEB) and hybridized to the membrane overnight at 37° C. in PerfectHyb™ Plus Hybridization Buffer (Sigma-Aldrich). The membrane was washed twice with 2×SSC buffer (Sigma-Aldrich), dried, and visualized by autoradiography using PhosphorImager (GE Healthcare).

### Western Blotting

Spermatozoa (~5 million cells) were lysed in 100 μl of 1× Laemmli sample buffer containing 10% 2-Mercaptoethanol by sonication. Proteins were separated using 12% SDS-PAGE and blotted onto a polyvinylidene difluoride membrane (GE Healthcare). The membrane was blocked in 5% milk/PBS for 1 h followed by overnight incubation with mouse monoclonal anti-TFAM antibody (Abcam, #ab119684, 1:1000 dilution) at 4° C. PolG was detected using anti-PolG antibody (Abcam, #128899, 1:1000). TEFM was detected using home-raised mouse monoclonal antibody against human TEFM (1:1000), while mtRNAP-using home-raised rabbit polyclonal antibodies against human A150 mtRNAP (1:1000). The membrane was washed three times with PBST (1× PBS and 0.1% Tween) and incubated with IRDye 680RD-labeled goat anti-mouse antibody (LI-Cor, #926-68070, 1:10,000 dilution) or IRDye 800CW-labeled goat anti-Rabbit IgG Secondary Antibody (LI-Cor, #926-32211, 1:15000) for 1 h at RT. The membrane was washed 6 times with PBST and imaged using the Infrared Imaging System Odyssey FC (LI-Cor).

### Cloning

For the lentiviral expression, the DNA region encoding TFAM-mScarlet fusion was excised from the pcDNA3-TFAM-mScarlet plasmid (Addgene ref #129573) using BamHI and EcoRI endonucleases and inserted into the pWPXL plasmid (Addgene ref #12257). To obtain the TFAM construct containing the 3′mitoUTR (3′mitoUTR TFAM-mScarlet_pWPXL), human heart cDNA (Zyagen, HD-801) was PCR-amplified using primers described in Table 2, and the amplicon cloned into a pT7Blue cloning vector (Novagen). Restriction sites (EcoRI/NdeI) were added through amplification by PCR to insert the fragment into the TFAM-mScarlet/pWPXL plasmid. The 5′mitoUTR was cloned into the TFAM-mScarlet/pWPXL or the 3′mitoUTR_TFAM-mScarlet_pWPXL plasmid using megaprimers following the QuikChange II XL Site-Directed Mutagenesis Kit (Agilent) large insertion protocol provided by the manufacturer. The Megaprimers for the 5′ mitoUTR were synthesized by amplifying DNA extracted from HEK293 cells using primers (Table 2). To obtain the TFAM constructs containing the 3′ and/or 5′ sperm TFAM UTRs, megaprimers were synthesized and cloned into the TFAM-mScarlet pWPXL plasmid following the QuikChange II XL Site-Directed Mutagenesis Kit (Agilent) large insertion protocol. The Megaprimers were synthesized by amplifying DNA extracted from HEK293 (Table 2). Deletion of the TFAM MTS (residues 1-42, 442TFAM-mScarlet) was performed using the QuikChange II XL Site-Directed Mutagenesis kit in 3′5′mitoUTR TFAM-mScarlet pWPXL.

TOM20 and H2B Type 1A ORFs were amplified by PCR from human heart cDNA (Zyagen). The amplicons were cloned into the pT7Blue cloning vector (Novagen) using the megaprimers (Table 2). The protocol for large insertions provided by the QuikChange II XL Site-Directed Mutagenesis Kit (Agilent) was followed to insert TOM20 or H2B in place of the TFAM gene TFAM-mScarlet_pWPXL plasmid. The MTS^{TFAM}mScarlet pWPXL construct was generated using QuikChange II XL site-directed mutagenesis kit by removing the TFAM sequence (residues 43-246) from the TFAM-mScarlet_pWPXL construct described above. To substitute the TFAM MTS with the mtRNAP MTS and generate the MTS^{Pol}Δ42TFAM-mScarlet construct, megaprimers were generated by PCR amplification of the MTS region (residues 1-45) of mtRNAP. The megaprimers were inserted into the TFAM-Scarlet_pWPXL plasmid.

Human mtRNAP (residues 1-1230) was cloned into pWPXL vector to obtain mtRNAP_pWPXL construct. Megaprimers containing the mScarlet gene were used to obtain the MTS^{Pol}-mScarlet-mtRNAP-pWPXL construct. The MTS^{pol}mScarlet_pWPXL construct was obtained by excising the MTS^{Pol}-mScarlet region using PmeI and XcmI and ligating this fragment into the pWPXL plasmid. Substitutions of the serine residues in the TFAM MTS (S31A/S34A) were performed using QuikChange II XL Site-Directed mutagenesis kit in the TFAM-mScarlet_pWPXL construct.

### Generation of Lentiviral Particles

In brief, 15 μg of the vector plasmid containing the gene of interest, 10 mg of the packaging construct plasmid psPAX2 (Adgene #12260), and 5 μg of VSV-G envelope expressing plasmid pMD2.G (Addgene #12259) were co-transfected into HEK293T cells using calcium phosphate transfection. The transfection mix was removed after 16 h and replaced with the complete media. The lentiviral particles were harvested after 24 and 48 h incubation, filtered through a 0.45 mm filter and concentrated by ultracentrifugation before being stored at −80° C. The infection efficiency of the viral particles was tested on HEK293T cells to ensure over 90% infection.

### Lentiviral Transduction of Human Somatic Cells and Spermatozoa

HeLa cells were seeded at a concentration of 415 cells/mm² in a 6-well plate to reach 90% confluency over 24 h before transduction. Upon incubating the lentiviral particles with HeLa cells for 16 h, the cells were washed twice with PBS and resuspended in a fresh complete culture medium. Transduced HeLa cells were obtained after 48 h post-transduction and resuspended in a fresh culture medium to be seeded for immunofluorescence (IF) staining. Viruses generated using the pWPXL vector were used as control.

To transduce the sperm cells, lentiviral particles were added to 1 ml of spermatozoa (~10⁷ cells) in a capacitation medium and incubated for 3-48 h. The transduced sperms were centrifuged at 600 g for 5 min, and the sperm pellet was resuspended in PBS to be further spotted on coverslips for IF staining.

### Immunofluorescence Staining

Before staining, HeLa cells were seeded on poly-L-lysine (Gibco) coated glass coverslips overnight, while human spermatozoa were spotted and air-dried directly on the coverslips after isolation. The samples were fixed with 4% paraformaldehyde (Thermo Fisher Scientific) for 20 minutes at RT, washed with PBS, permeabilized in PSB solution containing 0.25% Triton X-100 (Sigma-Aldrich) for 5 minutes, and blocked with PBS solution containing 1% BSA (Sigma-Aldrich) and 0.1% Triton X-100 for 1 h at 37° C. Followed by washing with PBS, the samples were blocked with 1% BSA-PBS solution for 1 h at 37° C. The prepared coverslips were incubated with the following primary antibodies prepared in mouse monoclonal anti-TFAM antibody (Abcam, #ab119684, 1:500) or monoclonal anti-TOM20 antibody (Cell Signaling Technology, #42406S, 1:500) in 0.1% Triton X-100 overnight at 4° C. The coverslips were washed three times with 0.1% Triton X-100 at 4° C. for 5 min, and the secondary antibody, donkey anti-mouse Alexa Fluor 555 (Abcam #ab 150110, 1:2000) or goat anti-rabbit Oregon Green 488 (Invitrogen #O-11038, 1:2000) was added for 2 h at RT. The coverslips were then counterstained with 300 nM DAPI (Invitrogen) and mounted with Prolong Glass antifade (Invitrogen). Digital images were obtained using a Nikon A1R+ confocal microscope at 60× magnification. The images were analyzed using the Fiji processing package on ImageJ.

### Transmission Electron Microscopy

Purified human spermatozoa were collected and fixed with freshly made fixative containing 3% paraformaldehyde and 0.1% glutaraldehyde in 0.1M phosphate buffer with 4% sucrose (pH 7.2). After washing with 0.1M phosphate buffer with 4% sucrose, 0.1M glycine was added to quench the unbonded aldehyde group. The cells were dehydrated in graded series of ethanol on ice and embedded in LR White (Electron Microscopy Sciences, Hatfield, PA). The samples were polymerized under UV light (360 nm) at −10° C. for 48 h, followed by 12 h at RT. The 90 nm-thin sections were cut and mounted on Formvar-Carbon coated 200 mesh nickel grids. The grid was incubated with an anti-TFAM antibody (1:1000, Abcam, #ab119684) in PBS with 1% BSA, and 0.05% Tween 20, for 2 h at RT, then overnight at 4° C. Upon washing, the anti-mouse secondary antibody conjugated with 18 nm gold (1:15, Colloidal Gold AffiniPure Goat Anti-Mouse IgG (H+L, EM Grade, Jackson ImmunoReasearch Laboratories, Inc.) were added in PBS with 1% BSA, and 0.05% Tween 20, and incubated for 1 h. After washing, the grids were stained with uranyl acetate and lead citrate by standard methods, imaged with Talos120C transmission electron microscope (Thermo Fisher Scientific), and recorded using Gatan (4k×4k) OneView Camera with software Digital Micrograph (Gatan Inc).

Measuring and Comparing Expression Levels of TEAM with Reference Levels

Spermatozoa were purified using a continuous one-step 90% gradient (ART-2100 and ART-1006; Sage) per the manufacturer's protocol. The cells were lysed by sonication in Laemmli buffer (ThermoFisher) and loaded into 12% SDS-PAGE (ThermoFisher). Separated proteins were blotted onto a polyvinylidene difluoride membrane (GE Healthcare). The membrane was blocked in 5% BSA in PBS (Life Technologies) for one hour, followed by overnight incubation with mouse monoclonal anti-TFAM antibody (Abcam, #ab119684, 1:1000 dilution) at 4° C. The membrane was washed three times with PBST (1× PBS and 0.1% Tween) and incubated with IRDye 680RD-labeled goat anti-mouse antibody (LI-Cor, #926-68070, 1:10,000 dilution) at room temperature for 1 hour. Upon incubation, the membrane is washed six times with PBST, and the fluorescent signal is detected using Infrared Imaging System Odyssey FC (LI-Cor).

Samples from individuals with fertile spermatozoa were inspected to reveal a single 30 kDa band representing the sperm TFAM isoform in a range of 30-100 ng per loaded sample (5 million cells), as revealed by using a reference recombinant TFAM of a known concentration. Samples revealing a second, 25 kDa band or exclusively a 25 kDa band on Western Blot or a significant decrease in the 30 kDa band intensity (>3-5 fold) may require supplementation of spermatozoa with the isolated sperm isoform TFAM during in vitro fertilization.

Assisted Reproductive Technology, i.e. Intracytoplasmic Sperm Injection (ICSI), Wherein TFAM is Added to the Fertilization Mix.

Human recombinant TFAM having modifications to improve its stability and mitochondrial import was produced in E. coli cells and purified according to the developed protocol by affinity chromatography. The protein was dissolved in PBS (Life Technologies), sterilized by filtration through 0.22 nm filters (Millipore), aliquoted, and stored at −86 C.

MII human oocytes were placed into a 50 μl micromanipulation droplet of TH3 medium with modified human tubal fluid (HTF, Irvine Scientific) and 10% HEPES. Spermatozoa were prepared by centrifugation (7 min, 200 g) of the liquid portion of the ejaculate and resuspending the sperm pellet in TH3 medium. Before final centrifugation and resuspension, an aliquot is taken to determine motility and concentration. Sperm concentrations were adjusted to 5×10⁶ spermatozoa per ml of TH3 medium and stored for three h at room temperature prior to ICSI. An oil-covered micromanipulation chamber having a 50 μl TH3 drop for oocytes and a 4 μl drop of 10% polyvinyl pyrrolidone (Irvine Scientific) supplemented with the TFAM solution for spermatozoa was prepared. The dish was then mounted on the stage of an inverted microscope (Olympus IX71) equipped with a stage warmer (TPi-SQX, Tokai Hit) and Narishige micromanipulators (MN-151, Narishige).

Individual spermatozoa were immobilized by compressing the tip of the tail against the lid of the dish with a microinjection pipette until a small bend in the middle of the flagellum is visible, aspirated into an ICSI pipette (Humagen) and injected into the oocyte cytoplasm, away from the polar body.

The injected oocytes were cultured in Global Medium (Life Global) with 10% serum substitute supplement (Global 10% medium) at 37° C. in 6% CO₂, 5% O₂, and 89% N₂. Fertilization is determined approximately 18 h after ICSI by noting the presence of two pronuclei and second polar body extrusion in the oocyte.

TABLE 1
Plasmids generated during the study

| Name of the construct | Parent plasmid |
|---|---|
| TFAM-mScarlet_pWPXL | pWPXL (Addgene ref #12257) |
| 3′mitoUTR_TFAM-mScarlet_pWPXL | TFAM-mScarlet_pWPXL |
| 5′mitoUTR_TFAM-mScarlet_pWPXL | TFAM-mScarlet_pWPXL |
| 3′5′mitoUTR_TFAM-mScarlet_pWPXL | 3′mitoUTR_TFAM-mScarlet_pWPXL |
| 3′nuclUTR_TFAM-mScarlet_pWPXL | TFAM-mScarlet_pWPXL |
| 5′nuclUTR_TFAM-mScarlet_pWPXL | TFAM-mScarlet_pWPXL |
| 3′5′nuclUTR_TFAM-mScarlet_pWPXL | 3′nuclUTR_TFAM-mScarlet_pWPXL |
| □42TFAM-mScarlet_pWPXL | 3′5′mitoUTR_TFAM-mScarlet_pWPXL |
| TOM20-mScarlet_pWPXL | TFAM-mScarlet_pWPXL |
| H2B-mScarlet_pWPXL | TFAM-mScarlet_pWPXL |
| MTS^{TFAM}mScarlet_pWPXL | TFAM-mScarlet_pWPXL |
| MTS^{pol}□42TFAM-mScarlet_pWPXL | TFAM-mScarlet_pWPXL |
| MTS^{pol}-mScarlet_pWPXL | mtRNAP_pWPXL |
| mScarlet_pWPXL | TFAM-mScarlet_pWPXL |
| S31A/S34A_TFAM-mScarlet_pWPXL | TFAM-mScarlet_pWPXL |
| TFAM-mScarlet_C-his6 | pcDNA-TFAM-mScarlet (Addgene ref # |
| | 129573) |

TABLE 2
Oligonucleotides used in the study

| | |
|---|---|
| Name of oligonucleotide | 5′-3′ Sequence |

TFAM: Cloning into pWPXL

| | |
|---|---|
| TFAM ORF END (FWD) | AAAATATGGTGCTGAGGAGTGTTAA |
| | (SEQ ID NO: 1) |
| TFAM 3′ Mt UTR (REV) | TGTGTTTCAACAAACTTTATTTATGAA |
| | CAC (SEQ ID NO: 2) |
| Restriction site: Mt 3′ UTR (FWD) | CCGGAATTCAAGTAGAAGATTGAGAT |
| | GTGTTCAC (SEQ ID NO: 3) |
| Restriction site: Mt 3′ UTR (REV) | GGAATTCCATATG TGTGTTTCAACAA |
| | ACTTTATTTATGAACAC(SEQ ID NO: 4) |
| Megaprimer: Nuclear 3′ UTR (FWD) | CGGCATGGACGAGCTGTACAAGTAAA |
| | AGTAGAAGATTGAGATGTGTTCAC |
| | (SEQ ID NO: 5) |
| Megaprimer: Nuclear 3′ UTR (REV) | ATCATATGACTAGTCCCGGGAATTCG |
| | AGAGAAAATAATTCAGAAAAAAATA |
| | AAATTCC (SEQ ID NO: 6) |
| Megaprimer: Mt 5′ UTR (FWD) | CCTCGAGGTTTAAACTACGGGATCCC |
| | CTCGCTAGTGGCGGGCATGATAACACA |
| | (SEQ ID NO: 7) |
| Megaprimer: Nuclear 5′ UTR (FWD) | CCTCGAGGTTTAAACTACGGGATCCG |
| | GGGTGAGGCCGCCGCCG |
| | (SEQ ID NO: 8) |
| Megaprimer: Mt/Nuclear 5′ UTR (REV) | CCCACATGCTTCGGAGAAACGCCAT |
| | (SEQ ID NO: 9) |
| Mutagenesis: Del42 TFAM-mScarlet with | CATCCACCGGAGCGATGTCATCTGTCT |
| Mt 5′ and 3′ UTRs (FWD) | TGGCAAGTTG (SEQ ID NO: 10) |
| Mutagenesis: Del42 TFAM-mScarlet with | CAACTTGCCAAGACAGATGACATCGC |
| Mt 5′ and 3′ UTRs (REV) | TCCGGTGGATG(SEQ ID NO: 11) |
| Megaprimer: Kozak mtRNAP MTS | GCCTCGAGGTTTAAACTACCATGTCG |
| (FWD) | GCACTTTGC(SEQ ID NO: 12) |
| Megaprimer: MTS RNAP TFAM (REV) | GGACAACTTGCCAAGACAGATGAGGC |
| | GGACGAGCTCCTCC(SEQ ID NO: 13) |
| Mutagenesis: MTS-Del TFAM Scarlet | CGAGGTGGTTTTCATCTGTCAGCAAG |
| (FWD) | GGCGAGGCAGTGAT(SEQ ID NO: 14) |
| Mutagenesis: MTS-Del TFAM Scarlet | ATCACTGCCTCGCCCTTGCTGACAGAT |
| (REV) | GAAAACCACCTCG(SEQ ID NO: 15) |
| Mutagenesis: TFAM S31D S34D (FWD) | GCTGTGGAAGTCGACTGCGCGACCCC |
| | TTCGATTTTGTGTATTTACCGAGGTG |
| | (SEQ ID NO: 16) |
| Mutagenesis: TFAM S31D S34D (REV) | CACCTCGGTAAATACACAAAATCGAA |
| | GGGGTCGCGCAGTCGACTTCCACAGC |
| | (SEQ ID NO: 17) |

TFAM: Cloning into pET3a

| | |
|---|---|
| Restriction site: Nde_TFAM (FWD) | GGAATTCCATATGGCGTTTCTCCGAA |
| | GCATGTGGG(SEQ ID NO: 18) |
| Restriction site: BamHI_mScarlet (REV) | CGCGGATCCTCTTACTTGTACAGCTCG |
| | TCCATGC(SEQ ID NO: 19) |
| Mutagenesis: HisTFAMscarletPET | GCATGGACGAGCTGTACAAGCATCAC |
| (FWD) | CATCACCATCACTAAGAGGATCCGGC |
| | TGC(SEQ ID NO: 20) |
| Mutagensis: HisTFAMscarletPET (REV) | GCAGCCGGATCCTCTTAGTGATGGTG |
| | ATGGTGATGCTTGTACAGCTCGTCCAT |
| | GC(SEQ ID NO: 21) |

Protein Controls: Cloning into pWPXL

| | |
|---|---|
| TOM20 FWD | ATGGTGGGTCGGAACAG |
| | (SEQ ID NO: 22) |
| TOM20 REV | TTCCACATCATCTTCAGCCA |
| | (SEQ ID NO: 23) |
| Megaprimer: TOM20 pt7blue (FWD) | CGAGGTTTAAACTACGGGATCCATGG |
| | TGGGTCGGAACAG |
| | (SEQ ID NO: 24) |
| Megaprimer: TOM20 pt7blue (REV) | CCTTGCTCACAGAACCACCACCACCTT |
| | CCACATCATCTTCAGCCA |
| | (SEQ ID NO: 25) |
| Megaprimer: H2B Type 1a (FWD) | CGAGGTTTAAACTACGGGATCCATGC |
| | CGGAGGTGTCATCT(SEQ ID NO: 26) |
| Megaprimer: H2B Type 1a (REV) | CCTTGCTCACAGAACCACCACCACCC |
| | TTGGAGCTGGTGTACTTAG |
| | (SEQ ID NO: 27) |

Northern Blot

| | |
|---|---|
| TFAM_Exon 4 | TCTTCTTTATATACCTGCCACTCCGCC |
| | CTA(SEQ ID NO: 28) |
| 18S rRNA | GCCCCGCGGGACACTCA |
| | (SEQ ID NO: 29) |

5′ & 3′ RLM-RACE, RT-PCR

| | |
|---|---|
| RNA Adaptor (Ambion) | GCUGAUGGCGAUGAAUGAACACUGC |
| | GUUUGCUGGCUUUGAUGAAA |
| | (SEQ ID NO: 30) |
| Adaptor_Inner (REV) | GTTCATTCATCGCCATCAGC |
| | (SEQ ID NO: 31) |
| Adaptor_Outer (REV) | TTTCATCAAAGCCAGCAAACGC |
| | (SEQ ID NO: 32) |
| Adaptor_Inner (FWD) (Ambion) | CGCGGATCCGAACACTGCGTTTGCTG |
| | GCTTTGATG(SEQ ID NO: 33) |
| Adaptor_Outer (FWD) (Ambion) | GCTGATGGCGATGAATGAACACTG |
| | (SEQ ID NO: 34) |
| TFAM Exon 3 (REV) | CCCTCCAACGCTGGGCAATT |
| | (SEQ ID NO: 35) |
| TFAM Exon 2 (REV) | CCAAGACAGATGAAAACCACC |
| | (SEQ ID NO: 36) |
| TFAM 3′ UTR (FWD) | AAGCCACGGTGTTCTGTGAT |
| | (SEQ ID NO: 37) |
| TFAM 3′ UTR Inner (FWD) | GCAGGCAGAACTCATCTAGG |
| | (SEQ ID NO: 38) |
| TFAM Exon 1T (FWD) | GGATTGCGGTTTCCCTTCAT |
| | (SEQ ID NO: 39) |

ddPCR: MtDNA

| | |
|---|---|
| mt92-CYTB (H-strand) | GGGTATAATTGTCTGGGTCGCC |
| | (SEQ ID NO: 40) |
| mt92-CYTB (L-strand) | AGACGCCCTCGGCTTACTTC |
| | (SEQ ID NO: 41) |
| mt64-ND1 (H-strand) | AGATGTGGCGGGTTTTAGGG |
| | (SEQ ID NO: 42) |
| mt64-ND1 (L-strand) | ACTACAACCCTTCGCTGACG |
| | (SEQ ID NO: 43) |

ddPCR: Nuclear DNA

| | |
|---|---|
| TEFM88 | GTGACTCCCGGACTAGTGGA |
| | (SEQ ID NO: 44) |
| TEFM88 | GATGGGAAGAACACCCGAGG |
| | (SEQ ID NO: 45) |
| TBP73 | CACCACAGCTCTTCCACTCA |
| | (SEQ ID NO: 46) |
| TBP73 | GGGGAGGGATACAGTGGAGT |
| | (SEQ ID NO: 47) |

### Example 1: Mature Human Spermatozoa Contain No Mitochondrial DNA

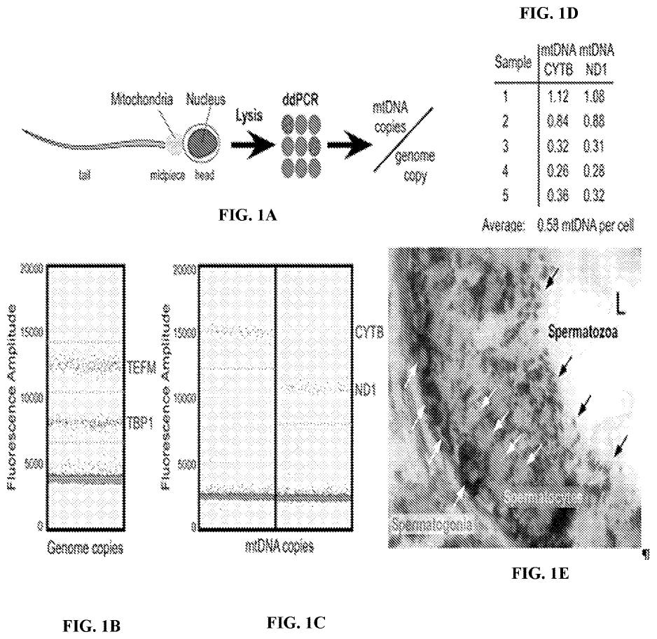

Previous studies reported a large variation of mtDNA copy number in a human spermatozoon—from 1 to 1000 genomes per cell. This discrepancy has been attributed to insufficient purification of sperm cells, inadequate primer design, or detection of chromosomal mtDNA-like sequences (NUMT). To accurately measure the mtDNA copy number in human sperm cells, Droplet Digital PCR (ddPCR) was used, which allows a determination of the absolute number of DNA molecules in a cell (FIG. 1A). Because the method does not require isolation of the genomic material, ddPCR is ideally suited for measuring low copy number nucleic acids. Amplification targeted two single-copy genes in the nuclear genome (TBP and TEFM) and two mitochondrial genes-CYTB and ND1 (FIGS. 1B, 1C). It was found that the sperm cells contain, on average, 0.58 copies of mtDNA (FIG. 1D). Each spermatozoon contains 50-70 mitochondria, corresponding to less than 0.01 mtDNA molecules per mitochondrion. While extremely low, this number likely accounts for the background mtDNA detected in a few contaminating cells (namely leucocytes), which can harbor up to 100 mtDNA per cell in their mitochondria.

Using an orthogonal strategy, in situ hybridization of mtDNA in human testicular tissue using RNAScope was performed (FIG. 1E). This method allows the detection of single DNA molecules per cell. An intense signal corresponding to mtDNA was detected in the mitochondria of spermatogonia, undifferentiated germ cells at the periphery of the seminiferous tubules (FIG. 1E). The primary spermatocytes, the cells developed from spermatogonium during the second stage of spermatocytogenesis, showed coarse staining of mtDNA, indicating its reduction during sperm maturation. Finally, mature spermatozoa had no detectible mtDNA signal in the midpiece region, in agreement with ddPCR analysis (FIG. 1E). It was concluded that the mature human spermatozoa are essentially devoid of mtDNA, consistent with maternal inheritance of the mitochondrial genome in mammals.

### Example 2: Lack of Transcription and Replication in Human Sperm Mitochondria

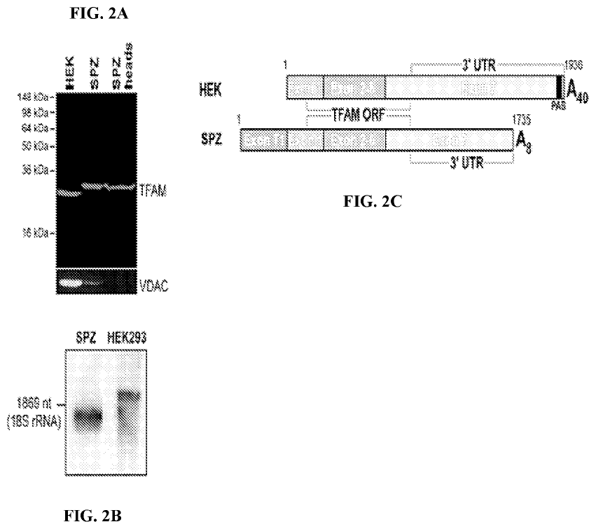

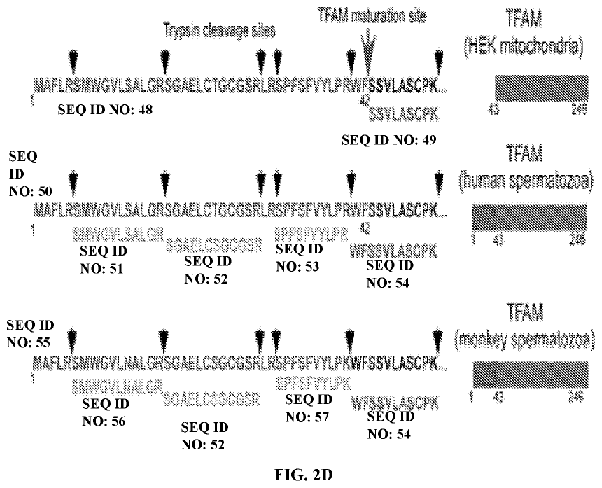

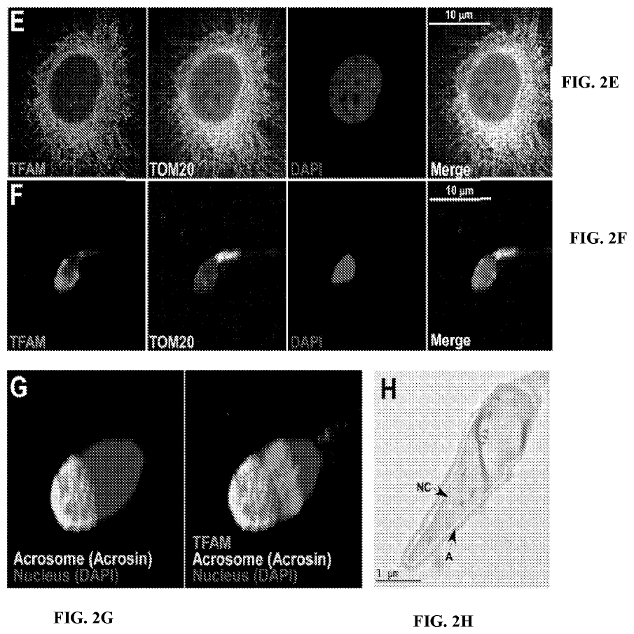

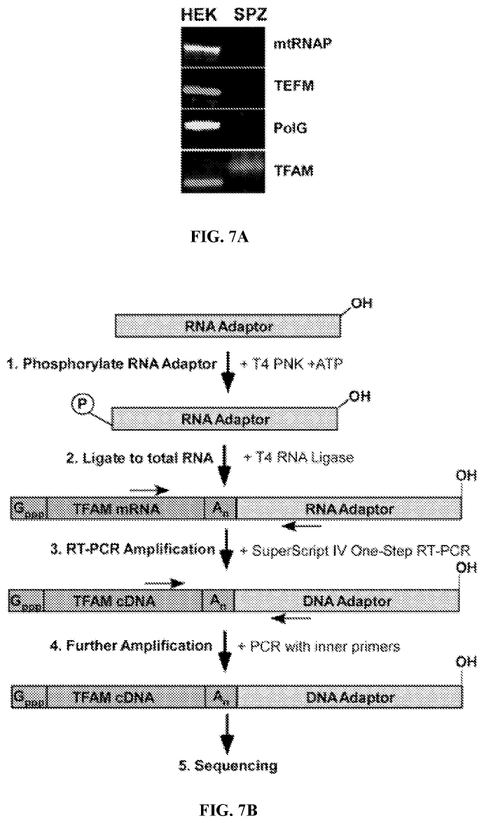

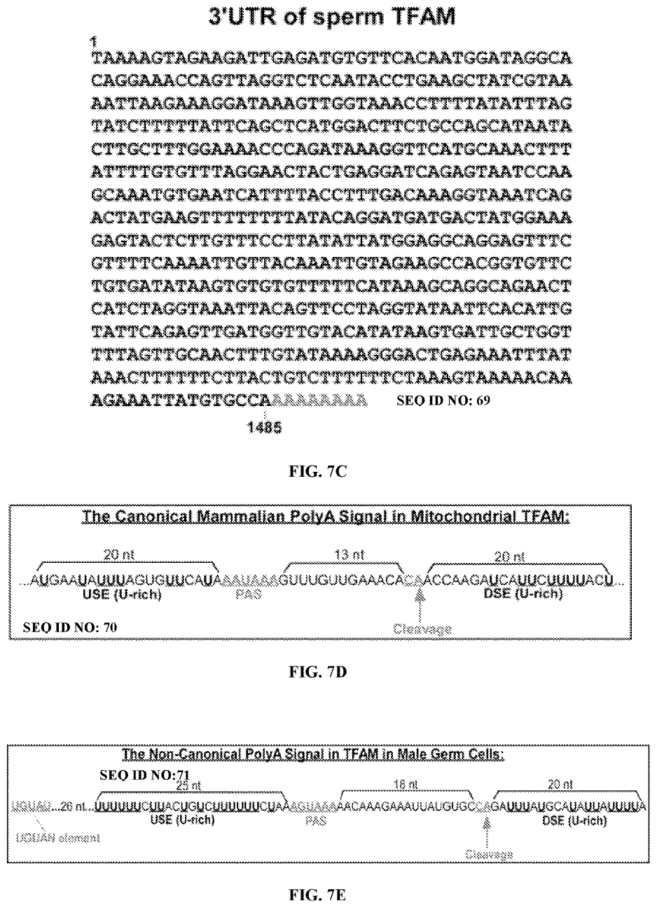

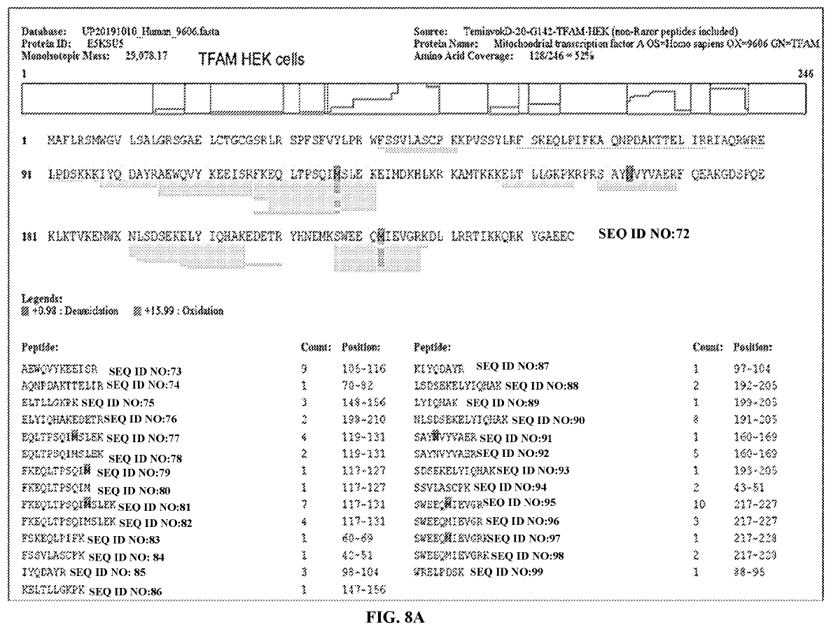

Next the presence of key proteins involved in transcription and replication of mtDNA in the mitochondria of human spermatozoa were looked into. It was found that human mitochondrial RNA polymerase (POLRMT), the catalytic subunit of DNA polymerase polG, and transcription elongation factor, TEFM, could not be detected by Western blotting (FIG. 7A). These proteins were also absent from the published complete spermatozoa proteome. These findings demonstrate that the spermatozoa mitochondria cannot maintain, replicate or transcribe mtDNA. Unexpectedly, it was found that human and monkey spermatozoa contain large amounts of the major mitochondrial nucleoid protein, transcription factor TFAM (FIGS. 2A-2H).

### Example 3: Human Spermatozoa Contain an Isoform of Mitochondrial Nucleoid Protein, TFAM

Western blot assays revealed a peculiar form of TFAM, which was ~5 kDa larger than the mature (mitochondrial) TFAM from HEK cells (FIG. 2A). A previous study detected alternative splicing of TFAM mRNA during the spermatogonia maturation. It was found that the mature sperm cells contain an alternatively spliced TFAM mRNA, not found in somatic cells (FIG. 2B).

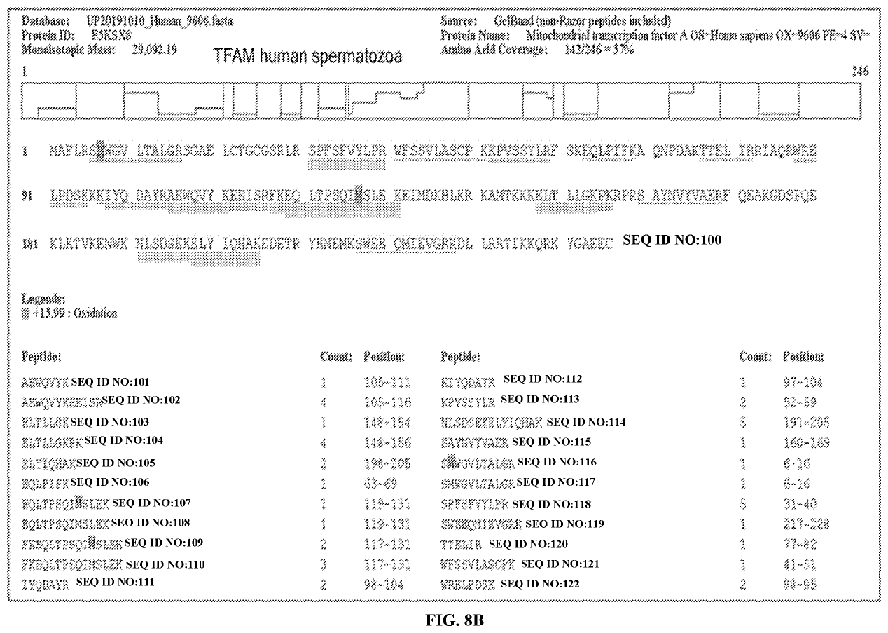

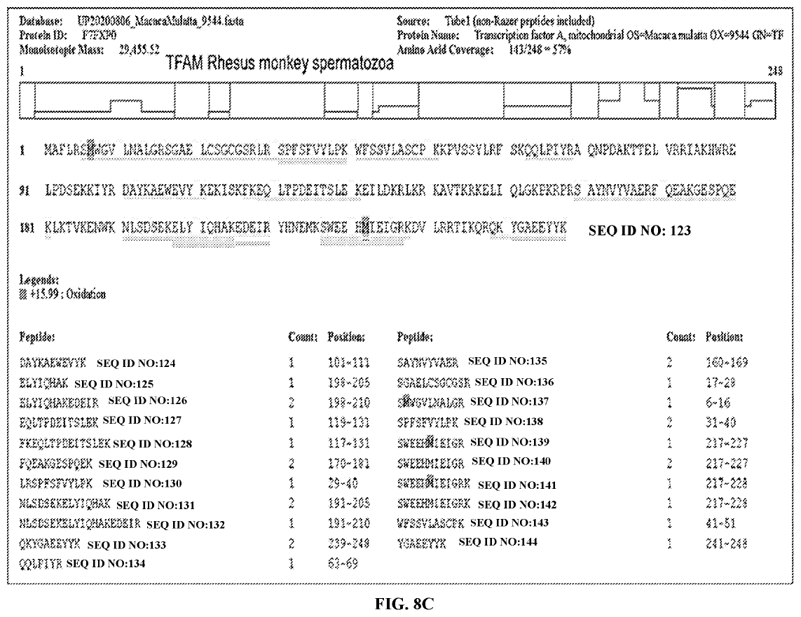

The 3′ UTR of this transcript was mapped and found that it is shorter than the one in somatic cells 30 and contains an abbreviated, ~8 nt long poly A-tail (FIG. 2C, FIG. 7C). In agreement with the previous data obtained for testis tissue, as the result of an alternative splicing event, the sperm TFAM mRNA features an additional exon T1 (FIG. 2B). However, the T1 exon does not alter the protein open reading frame, and thus the translation of the sperm mRNA isoform results in the synthesis of a full-size TFAM precursor (residues 1-246), as confirmed by LC-/MS/MS analysis (FIG. 2D, FIGS. 8A-8C). Both human and Rhesus monkey sperm TFAM contain the peptides found in the mitochondrial targeting sequence (residues 1-42) but lack a partial tryptic fragment (evidence of protein maturation), suggesting that unlike the mitochondrial TFAM in 5 somatic cells, this protein isoform does not undergo mitochondrial processing, in which this signal would be removed (FIG. 2D).

### Example 4: TFAM is Found in the Nucleus but not in the Mitochondria of the Sperm Cells

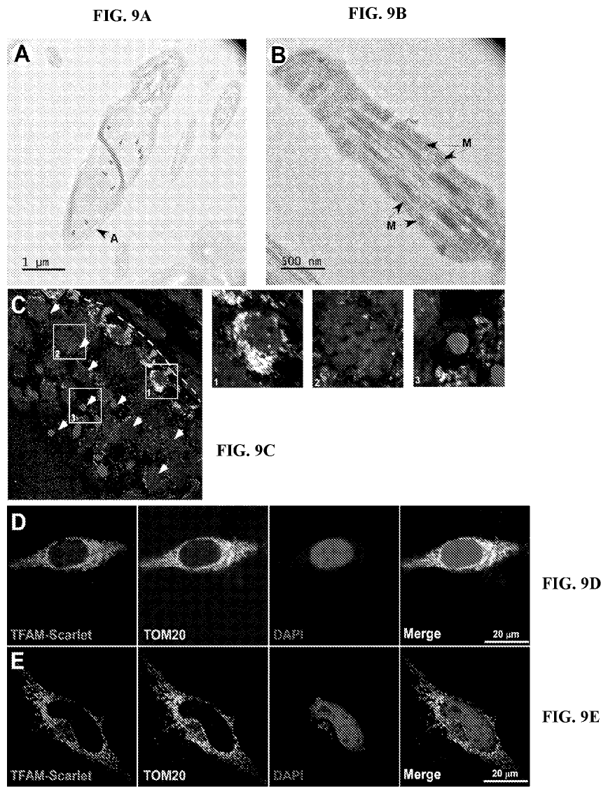

TFAM localizes exclusively to the mitochondrial reticulum in somatic cells (FIG. 2E). In contrast, confocal microscopy using an anti-TFAM antibody reveals TFAM in the head of spermatozoa but not in the mitochondria at the midpiece region (FIG. 2F). This is consistent with the presence of an unprocessed mitochondrial targeting sequence in the sperm TFAM isoform, as established by LCMS/MS analysis. Since TFAM is absolutely required for mtDNA maintenance, replication, and transcription, and reduction of its expression is linked to mtDNA elimination in all animal models tested, the lack of TFAM in the spermatozoa mitochondria explains why these cells do not possess mtDNA. Confocal microscopy revealed a nuclear localization of TFAM in the anterior half of the spermatozoa head, in proximity to the acrosome, a sperm-specific organelle containing enzymes required to lyse corona radiata and zona pellucida of the oocyte during fertilization (FIG. 2F). However, a detailed analysis of TFAM localization by layered confocal microscopy followed by 3D reconstruction of the deconvolved images suggests a clear co-localization of TFAM with the spermatozoon nucleus and not with the acrosome (FIG. 2G). Further, immunogold staining of TFAM and transmission cryogenic electron microscopy analysis revealed the presence of gold stained TFAM in the nucleus of the sperm cells (FIG. 2H) but not in the sperm mitochondria, as evident from the analysis of the images taken (n=25) (FIGS. 9A-9B).

### Example 5: The Mitochondrial Targeting Signal (MTS), but not UTRs, Affects TFAM Trafficking into Cells' Mitochondria or Nucleus

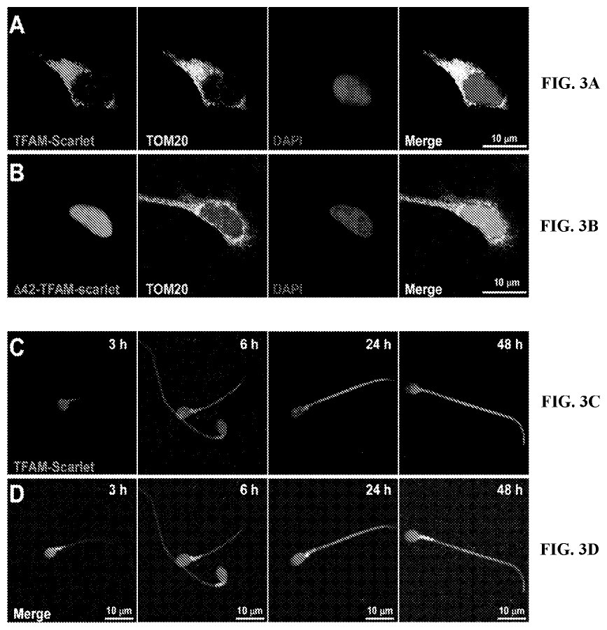

Cytosolic translation of some yeast mitochondrial proteins is mediated by PUF proteins that recognize the 3′UTRs sequences and direct the mRNAs to the ribosomes associated with the mitochondrial outer membrane to enable a co-translational import. Assuming that a similar targeting system might function in other eukaryotes and considering that an alternatively spliced variant of TFAM mRNA has been found in sperm cells, it was sought to investigate how the presence of the sperm-specific UTRs affects the trafficking of TFAM in spermatozoa. Transduction of HeLa cells with lentiviral constructs containing TFAM mRNA with either the sperm-specific (nuclear) or somatic (mitochondrial) 5′- and 3′-UTRs, or the mRNA lacking the UTRs altogether resulted in TFAM localization exclusively into the mitochondria, as evident by confocal microscopy (FIG. 3A, FIGS. 9D, 9E). In contrast, when the mitochondrial pre-sequence has been deleted, TFAM was no longer detected in the mitochondria of HeLa cells but instead localized to the nucleus (FIG. 3B). It was speculated that the nuclear localization of TFAM lacking mitochondrial pre-sequence results from a predicted nearly-consensus bipartite nuclear localization signal in TFAM.

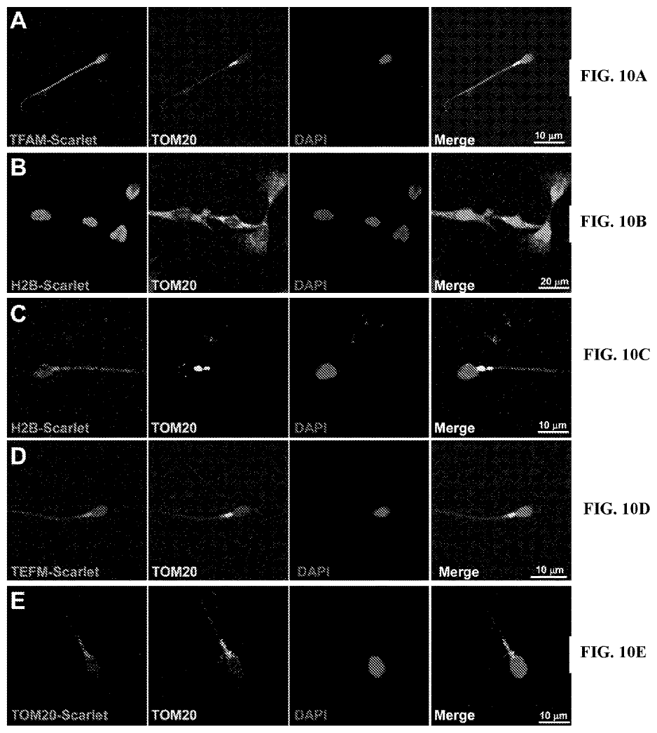

To investigate TFAM trafficking in sperm cells, lentiviral transduction experiments were conducted (FIG. 3C). An efficient accumulation of TFAM-mScarlet in the cytoplasm, visible by a progression of the red fluorescent signal from the sperm head to its tail, was observed (FIG. 3C). Interestingly, no TFAM accumulation was detected in the nucleus or mitochondria (FIG. 3D). TFAM was localized in the cytoplasm of the sperm cells irrespective of the presence or absence of UTRs (FIG. 10A). The extremely high packing density of the genomic material, about 10-fold higher than in somatic cells, could explain the absence of exogenous TFAM in the sperm nucleus. Indeed, overexpression of the sperm nuclear protein, histone H2B, shows cytoplasmic localization of this protein in sperm cells but nuclear localization in somatic cells (FIGS. 10B, 10C). Notwithstanding, the import of proteins into sperm mitochondria appears active, as mitochondrial localization of the overexpressed TOM20 in the transduced spermatozoa was detected (FIGS. 10D, 10E). Most importantly, the lack of TFAM-mScarlet fluorescence signal in mitochondria of the transduced sperm cells suggests that the import of the over-expressed protein is being actively prevented.

### Example 6: Phosphorylation of TFAM Pre-Sequence Prevents its Import into the Mitochondria of Sperm Cells

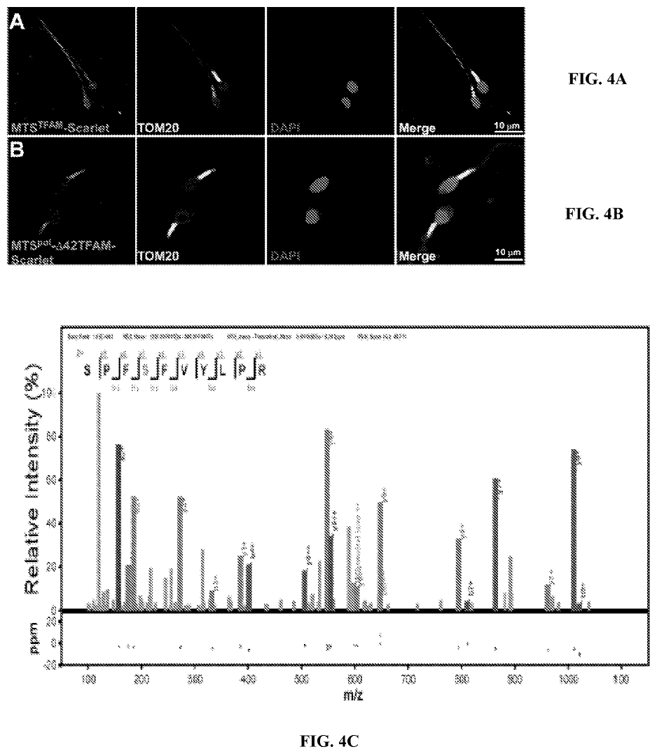

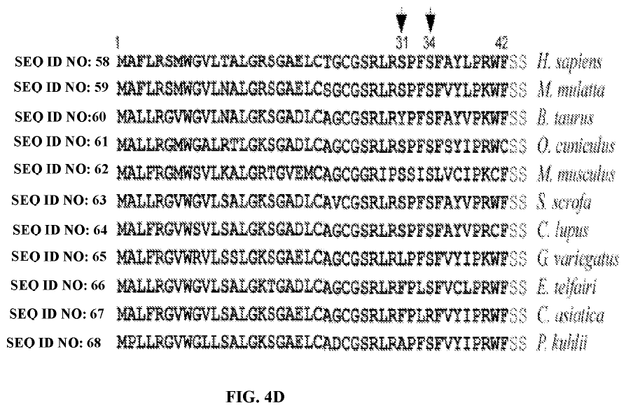

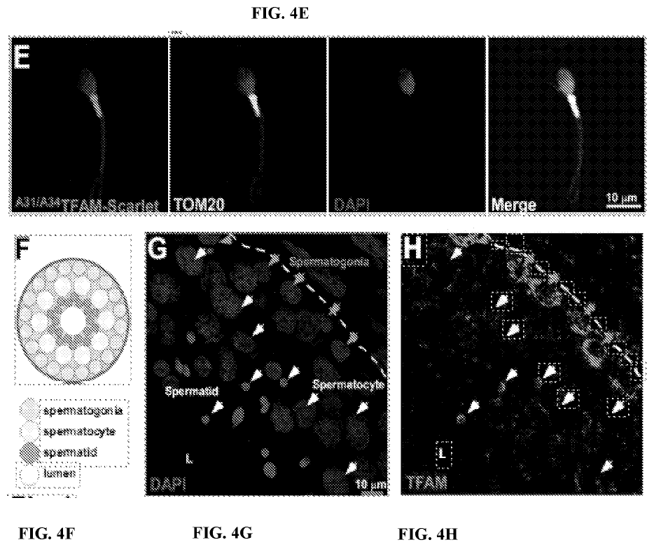

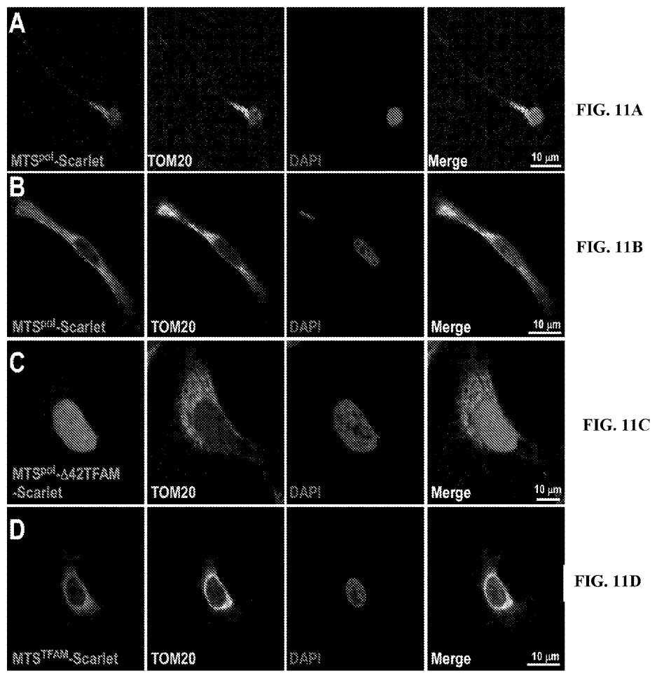

To understand the reason behind the lack of TFAM import into spermatozoa mitochondria, the cells with lentivirus carrying red fluorescent protein mScarlet were fused to the mitochondrial pre-sequence of TFAM or of mtRNAP (FIG. 4A, FIGS. 11A-11D). While mScarlet localization was observed in sperm mitochondria when mtRNAP pre-sequence was used (FIG. 11A), no fluorescence signal was detected inside mitochondria in the case of the TFAM pre-sequence (FIG. 4A), hinting at the key role of this region in sperm TFAM localization. Indeed, when D42-TFAM-mScarlet fusion with the mtRNAP pre-sequence was used, this protein was localized to spermatozoa mitochondria (FIG. 4B). Discovering the crucial role of the pre-sequence in preventing the mitochondrial import of TFAM prompted the search for posttranslational modifications in this region. LC-MS/MS analysis of human sperm TFAM identified phosphorylation of the residue S34 in the pre-sequence (FIG. 4C). Analysis of the human sperm phosphoproteome confirmed the presence of phosphorylation at S34 and, in addition, at S31 in the sperm TFAM pre-sequence. Recognition of the mitochondrial pre-sequences is sequence-independent and is based on their secondary structure (commonly a helix) and the positive charge of this region. Phosphorylation of S31 and S34, conserved in mammalian TFAM (FIG. 4D), is expected to bring a large negative net charge to the pre-sequence region and prevent mitochondrial import. More than 40% of MTS-containing mitochondrial proteins are phosphorylated within or proximal 15 to this sequence. While the function of MTS phosphorylation is largely unknown, several reports indicate inhibition of protein translocation into mitochondria. To confirm that the phosphorylation of the TFAM pre-sequence region plays a role in preventing mitochondrial import of TFAM in spermatozoa, S31 and S34 were substituted residues with alanines and transduced sperm cells with lentivirus carrying this variant. It was found that TFAMS31A/S34A was localized exclusively to the sperm mitochondria, confirming the role of phosphorylation in preventing mitochondrial localization of TFAM in mature spermatozoa (FIG. 4E).

### Example 7: Re-Localization of the Sperm TFAM During Spermatogenesis

The data suggest that during sperm cell development, TFAM should switch its localization from mitochondria to the nucleus. Human testicular tissue was stained with an anti-TFAM antibody (FIGS. 4F-4H). An intense TFAM signal was detected in the mitochondria of spermatogonia, undifferentiated germ cells at the periphery of the seminiferous tubules (FIG. 4H). In contrast, primary spermatocytes, the cells developed from spermatogonium during the second stage of spermatocytogenesis and distinguished by the coarse chromatin pattern, showed only traces of TFAM, which partially overlaps with TOM20, indicative of mitochondrial localization (FIG. 9C). Closer to the lumen, the immature sperm cells-round spermatids-reveal intense DAPI staining of their compacted nuclei, which co-localized with the TFAM signal (FIG. 4H). Because the second meiotic stage is short and only a few, if any, secondary spermatocytes are present in the tissue, it could not be determined if TFAM is present in the nuclei of these cells. It was speculated that TFAM is already present in the nuclei of the primary spermatocytes but becomes visible only when “concentrated” by the nuclear condensation in spermatids. Remarkably, the re-location of TFAM from the mitochondria of immature sperm cells to the spermatozoa nucleus coincides with the disappearance of mtDNA observed during spermatogenesis (FIG. 1E).

### Example 8: Transfer of the Sperm TFAM into Oocyte During Fertilization

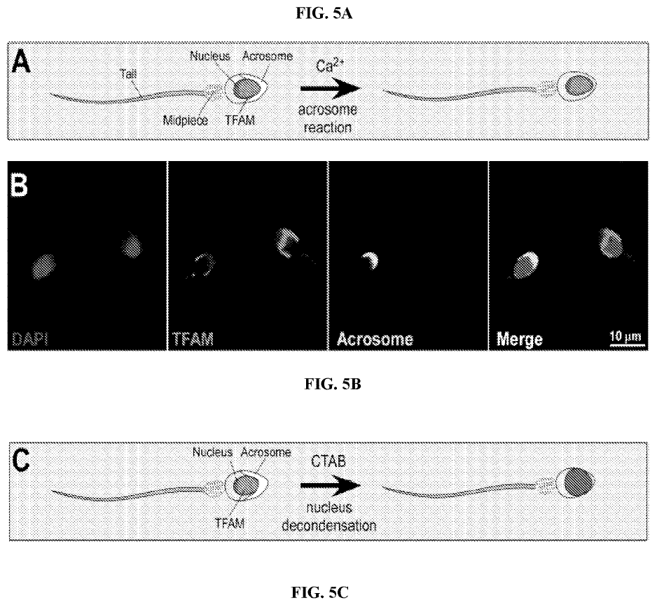

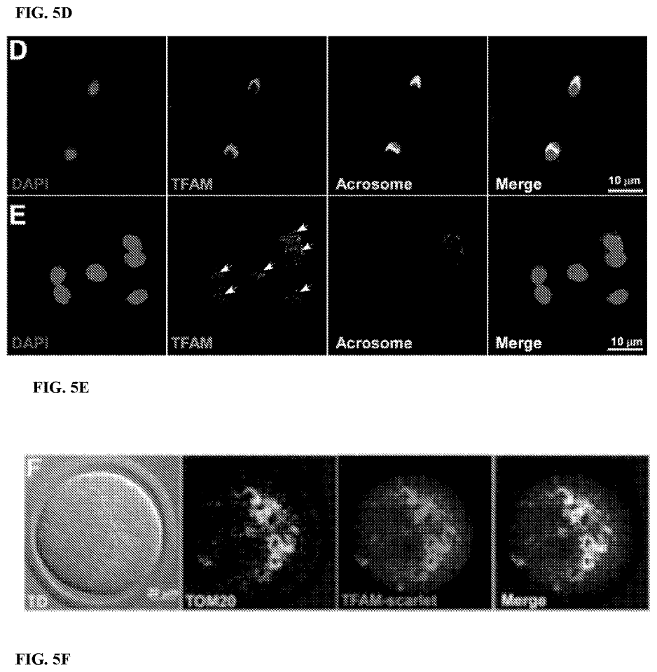

The switch in TFAM localization from mitochondria to the nucleus due to phosphorylation of the TFAM pre-sequence during sperm maturation could serve two functions. First, eliminating TFAM from the sperm mitochondria leaves the mtDNA unprotected, causing its efficient elimination and precluding its transmission into a zygote. Second, preventing TFAM import into mitochondria results in the accumulation of large quantities of TFAM in the spermatozoa nucleus. Indeed, estimates suggest that a mature human spermatozoon harbors 0.3-0.5 million TFAM molecules. What will happen with this massive amount of TFAM during fertilization? At the initial stages of fertilization, spermatozoa undergo the acrosome reaction, during which the membrane covering the acrosome ruptures on multiple sperm cells attached to the oocyte. This process releases hydrolytic enzymes necessary to make holes in zona pellucida and help a sperm cell penetrate the oocyte. The acrosome reaction can be stimulated in vitro in the presence of Ca2⁺ ions (FIG. 5A). It was found that during the acrosome reaction, the acrosome is lost, while TFAM remains in the sperm nucleus (FIG. 5B). This suggests that the sperm TFAM likely enters the oocyte cytoplasm, carried by the densely packed genomic material upon fertilization. Prior to the male pronucleus formation, the sperm nucleus must undergo the process of decondensation (FIG. 5C), during which the enlargement of the compact sperm nucleus is observed. Stimulating the sperm nuclei de-condensation in vitro by incubating the cells with a mild detergent, CTAB results in a noticeable expansion of the genomic material (FIGS. 5D-5E). Unlike the situation with the sperm histone protein H2B, which remains bound to DNA during de-condensation, the sperm TFAM was efficiently dispersed, as evident by the detection of only trace amounts of TFAM in the sperm nucleus (FIG. 5E). This suggests that TFAM may be released into the oocyte cytoplasm during sperm nuclei de-condensation.

### Example 9: Sperm TFAM can be Delivered into Oocyte Mitochondria

Since the sperm, or paternal, TFAM is delivered to the oocyte during fertilization, what possible fate is expected for this protein? In principle, the paternal TFAM is indistinguishable from the maternal TFAM synthesized in oocytes, except for the presence of the labile phosphorylated residues in the pre-sequence region. Because of the TFAM pre-sequence, it is expected that the import machinery in oocyte mitochondria will recognize TFAM and deliver it to maternal mitochondria. To prove this, the sperm TFAM-mScarlet fusion protein was generated, purified and used in microinjection experiments involving mature mouse metaphase II oocytes (FIG. 5F). Upon injection of ~0.5 million TFAM molecules into an oocyte and subsequent incubation, the oocytes were stained with an anti-TOM20 antibody to reveal the mitochondria. The red fluorescent signal accumulation was detected in oocyte mitochondria, illustrating how the sperm TFAM can be efficiently delivered into these organelles (FIG. 5F).

### Example 10: The Possible Role of Paternal TFAM in Fertilization

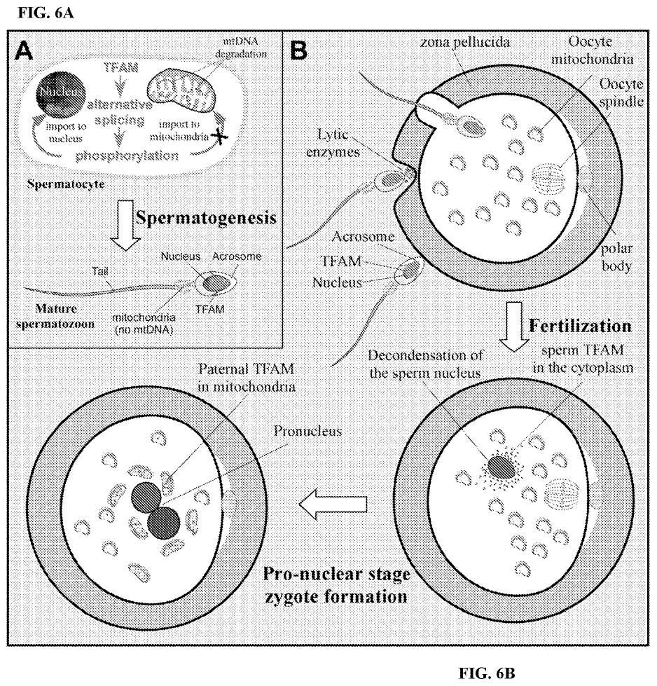

Maternal inheritance of mtDNA is a major paradigm that guides the existence and evolution of the vast majority of species. Based on the data, a model in which the paternal contribution of sperm TFAM to the zygote mitochondria plays a key role in a mechanism of quality control of fertilization is proposed. The model is based on three key findings. First, it was demonstrated that the major mitochondrial protein TFAM is not found in sperm mitochondria, explaining the lack of mtDNA in these cells (FIG. 6A). Second, contrary to the other key mitochondrial replication and transcription proteins, TFAM expression has not been shut down during spermatogenesis. Instead, TFAM synthesis has been maintained at a high level, likely due to alternative splicing resulting in a shorter 3′UTR and alternative polyadenylation, and the protein re-directed from mitochondria to the sperm nucleus. The switch in TFAM localization from the mitochondria of primary sperm cells into the nucleus of mature spermatozoa during spermatogenesis coincides with the degradation of mtDNA (FIG. 6A). Finally, despite the alternative splicing of TFAM premRNA, the protein's mitochondrial targeting signal has been preserved, hinting at its import into oocyte mitochondria and its role in fertilization. Taken together, these findings suggest that there is a quality control mechanism to prevent the transfer of the paternal mtDNA into the oocyte and ensure that fertilization of an egg is possible only by a spermatozoon that does not carry amitochondrial genome (FIG. 6B). It was hypothesized that the presence of TFAM in the sperm nucleus communicates to an oocyte that the partner cell lacks mtDNA and is suitable for fertilization (FIG. 6B). Transmission electron micrography experiments revealed that human oocyte's mitochondria are small and round and have underdeveloped “arched” cristae. Such cristae do not harbor as many OXPHOS complexes as in somatic mitochondria, consistent with the finding that only a small number of oocyte mitochondria are metabolically active. It is, therefore, tempting to speculate that the large amount of the paternal TFAM delivered into oocyte mitochondria might stimulate the zygote's mtDNA transcription and replication (FIG. 6B). These processes, in turn, can boost oxidative phosphorylation and supply the energy required for zygote development. The abnormal spermatozoa carrying mitochondria may not have sufficient amounts of TFAM in the nucleus. They will fail to stimulate zygote formation, eliminating the risk of a paternal mtDNA transfer. The phenomenon of paternal TFAM transfer reported here has important implications for the fields of human fertility and germ cell therapy. Spermatozoa that are deficient in nuclear TFAM could account for unexplained male infertility. Indeed, elevated mtDNA levels were found in sperm of men with severe oligoasthenospermia. These findings suggest that sperm TFAM is as a potential biomarker of male infertility. The import of TFAM to the oocyte mitochondria could result in dramatic changes in the transcription and replication status of the zygote's mitochondria. The proposed quality control mechanism, which ensures the maternal inheritance of mtDNA in mammalian species, a key evolution trait, is based upon an elegant molecular interplay.

### Enumerated Embodiments

The following exemplary embodiments are provided, the numbering of which is not to be construed as designating levels.

Embodiment 1 comprises a method of providing an assisted reproduction treatment to an infertile or a hypofertile subject, wherein the method comprises supplementing spermatozoa of the subject with an effective amount of recombinant transcription factor A, mitochondrial (TFAM) during fertilization.

Embodiment 2 provides the method of embodiment 1, wherein the infertile or the hypofertile subject is the subject in whom the expression level and/or molecular weight of sperm TFAM is/are reduced relative to the expression level and/or the molecular weight of sperm TFAM from a reference, wherein the reference comprises spermatozoa from a fertile subject.

Embodiment 3 provides the method of embodiments 1-2, wherein the reference expresses about 300,000-500,000 TFAM molecules per spermatozoon.

Embodiment 4 provides the method of embodiments 1-3, wherein the reference expresses about 30 ng to about 100 ng of sperm TFAM per five million spermatozoa.

Embodiment 5 provides the method of embodiments 1-4, wherein the reference sperm TFAM has a molecular weight of about 30 kDa.

Embodiment 6 provides the method of embodiments 1-5, wherein the assisted reproduction treatment comprises Intracytoplasmic Sperm Injection (ICSI) treatment, and wherein the supplementing comprises injecting the recombinant TFAM into an oocyte.

Embodiment 7 provides a method of identifying an infertile or a hypofertile subject, the method comprising
a) determining the expression level and/or the molecular weight of sperm TFAM in a spermatozoa sample prepared from semen obtained from the subject, and
b) comparing the expression level and/or the molecular weight of sperm TFAM from the sample with the expression level and/or the size of sperm TFAM from a reference, wherein lower expression level and/or reduced molecular weight of sperm TFAM from the sample relative to the expression level and/or the molecular weight of sperm TFAM from the reference indicates that the subject is infertile or hypofertile.

Embodiment 8 provides the method of embodiment 7, wherein the reference comprises spermatozoa from a fertile subject.

Embodiment 9 provides the method of embodiments 7-8, wherein the reference expresses about 300,000-500,000 TFAM molecules per spermatozoon.

Embodiment 10 provides the method of embodiments 7-9, wherein the reference expresses about 30 ng to about 100 ng of sperm TFAM per five million spermatozoa.

Embodiment 11 provides the method of embodiments 7-10, wherein the reference sperm TFAM has a molecular weight of about 30 kDa.

Embodiment 12 provides the method of embodiments 7-11, wherein the expression level and/or the molecular weight of sperm TFAM is determined using at least one selected from the group consisting of Western blot, enzyme-linked immunosorbent assay (ELISA), mass spectroscopy, and confocal microscopy.

Embodiment 13 provides a method of improving fertility of a spermatozoa of an infertile or a hypofertile subject for an assisted reproduction treatment, the method comprising:
a. determining the expression level and/or the molecular weight of sperm TFAM in a spermatozoa sample prepared from semen obtained from the subject;
b. comparing the expression level and/or the molecular weight of sperm TFAM from the sample with the expression level and/or the molecular weight of sperm TFAM from a reference, wherein lower expression level and/or reduced molecular weight of sperm TFAM from the sample relative to the expression level and/or the molecular weight of sperm TFAM from the reference indicate that the spermatozoa sample is from the infertile or the hypofertile subject, wherein the reference comprises spermatozoa from a fertile subject; and
c. supplementing the infertile or the hypofertile subject's spermatozoa with an effective amount of recombinant TFAM during fertilization to improve its fertility.

Embodiment 14 provides the method of embodiment 13, wherein the expression level and/or the molecular weight of sperm TFAM is determined using at least one selected from the group consisting of Western blot, enzyme-linked immunosorbent assay (ELISA), mass spectroscopy, and confocal microscopy.

Embodiment 15 provides a method of diagnosing and treating an infertile or a hypofertile subject, the method comprising
a. identifying the infertile or the hypofertile subject, wherein the identifying comprises the steps of:
   determining the expression level and/or the molecular weight of sperm TFAM in a spermatozoa sample prepared from semen obtained from the subject, and
   comparing the expression level and/or the molecular weight of sperm TFAM from the sample with the expression level and/or the size of sperm TFAM from a reference, wherein lower expression level and/or reduced molecular weight of sperm TFAM from the sample relative to the expression level and/or the molecular weight of sperm TFAM from the reference indicates that the subject is infertile or hypofertile, wherein the reference comprises spermatozoa from a fertile subject; and
b. providing assisted reproduction treatment to the subject identified as infertile or hypofertile, wherein the assisted reproduction treatment comprises Intracytoplasmic Sperm Injection (ICSI) treatment, wherein spermatozoa of the subject is supplemented with an effective amount of recombinant TFAM during fertilization.

Embodiment 16 provides a composition for improving fertility of spermatozoa of an infertile or a hypofertile subject, wherein the composition comprises a fertilization mix for ICSI, wherein the fertilization mix includes oocytes in Tyrode albumin lactate pyruvate medium (TH3) and an effective amount of recombinant TFAM, wherein the composition further includes spermatozoa for injecting into the oocytes.

Embodiment 17 provides a kit comprising reagents for ICSI procedure, wherein the kit comprises Tyrode albumin lactate pyruvate medium (TH3), modified human tubal fluid, a recombinant TFAM solution, and an instructional material for use thereof.

Embodiment 18 provides the method or the composition or the kit of embodiments 1-17, wherein the subject is a mammalian male.

Embodiment 19 provides the method or the composition or the kit of embodiments 1-18, wherein the subject is a human male.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of providing an assisted reproduction treatment to an infertile or a hypofertile subject, wherein the method comprises supplementing spermatozoa of the subject with an effective amount of recombinant transcription factor A, mitochondrial (TFAM) during fertilization.

2. The method of claim 1, wherein the infertile or the hypofertile subject is a subject in whom the expression level and/or molecular weight of sperm TFAM is/are reduced relative to the expression level and/or the molecular weight of sperm TFAM from a reference, wherein the reference comprises spermatozoa from a fertile subject.

3. The method of claim 2, wherein the reference expresses about 300,000-500,000 TFAM molecules per spermatozoon.

4. The method of claim 2, wherein the reference expresses about 30 ng to about 100 ng of sperm TFAM per five million spermatozoa.

5. The method of claim 2, wherein the reference sperm TFAM has a molecular weight of about 30 kDa.

6. The method of claim 1, wherein the assisted reproduction treatment comprises Intracytoplasmic Sperm Injection (ICSI) treatment, and wherein the supplementing comprises injecting the recombinant TFAM into an oocyte.

7. A method of identifying an infertile or a hypofertile subject, the method comprising
a) determining the expression level and/or the molecular weight of sperm TFAM in a spermatozoa sample prepared from semen obtained from the subject, and
b) comparing the expression level and/or the molecular weight of sperm TFAM from the sample with the expression level and/or the size of sperm TFAM from a reference, wherein a lower expression level and/or reduced molecular weight of sperm TFAM from the sample relative to the expression level and/or the molecular weight of sperm TFAM from the reference indicates that the subject is infertile or hypofertile.

8. The method of claim 7, wherein the reference comprises spermatozoa from a fertile subject.

9. The method of claim 7, wherein the reference expresses about 300,000-500,000 TFAM molecules per spermatozoon.

10. The method of claim 7 wherein the reference expresses about 30 ng to about 100 ng of sperm TFAM per five million spermatozoa.

11. The method of claim 7, wherein the reference sperm TFAM has a molecular weight of about 30 kDa.

12. The method of claim 7, wherein the expression level and/or the molecular weight of sperm TFAM is determined using at least one selected from the group consisting of Western blot, enzyme-linked immunosorbent assay (ELISA), mass spectroscopy, and confocal microscopy.

13. A method of improving fertility of a spermatozoa of an infertile or a hypofertile subject for an assisted reproduction treatment, the method comprising:
a. determining the expression level and/or the molecular weight of sperm TFAM in a spermatozoa sample prepared from semen obtained from the subject;
b. comparing the expression level and/or the molecular weight of sperm TFAM from the sample with the expression level and/or the molecular weight of sperm TFAM from a reference, wherein a lower expression level and/or reduced molecular weight of sperm TFAM from the sample relative to the expression level and/or the molecular weight of sperm TFAM from the reference indicates that the spermatozoa sample is from the infertile or the hypofertile subject, and wherein the reference comprises spermatozoa from a fertile subject; and
c. supplementing the infertile or the hypofertile subject's spermatozoa with an effective amount of recombinant TFAM during fertilization to improve its fertility.

14. The method of claim 13, wherein the expression level and/or the molecular weight of sperm TFAM is determined using at least one selected from the group consisting of Western blot, enzyme-linked immunosorbent assay (ELISA), mass spectroscopy, and confocal microscopy.

15. A method of diagnosing and treating an infertile or a hypofertile subject, the method comprising
a. identifying the infertile or the hypofertile subject, wherein the identifying comprises the steps of:
determining the expression level and/or the molecular weight of sperm TFAM in a spermatozoa sample prepared from semen obtained from the subject,
comparing the expression level and/or the molecular weight of sperm TFAM from the sample with the expression level and/or the size of sperm TFAM from a reference, wherein a lower expression level and/or reduced molecular weight of sperm TFAM from the sample relative to the expression level and/or the molecular weight of sperm TFAM from the reference indicates that the subject is infertile or hypofertile, wherein the reference comprises spermatozoa from a fertile subject; and
b. providing assisted reproduction treatment to the subject identified as infertile or hypofertile, wherein the assisted reproduction treatment comprises Intracytoplasmic Sperm Injection (ICSI) treatment, wherein spermatozoa of the subject is supplemented with an effective amount of recombinant TFAM during fertilization.

16. A composition for improving fertility of spermatozoa of an infertile or a hypofertile subject, wherein the composition comprises a fertilization mix for ICSI, wherein the fertilization mix includes oocytes in Tyrode albumin lactate pyruvate medium (TH3) and an effective amount of recombinant TFAM, wherein the composition further includes spermatozoa for injecting into the oocytes.

17. A kit comprising reagents for ICSI procedure, wherein the kit comprises Tyrode albumin lactate pyruvate medium (TH3), modified human tubal fluid, a recombinant TFAM solution, and an instructional material for use thereof.

18. The method of claim 1, wherein the subject is a mammalian male.

19. The method of claim 18, wherein the subject is a human male.


SEQUENCE LISTING (144 sequences)

SEQ ID NO: 1 - DNA, 25 bp, synthetic construct
  misc_feature 1..25: note = primer
  source 1..25: mol_type = other DNA
aaaatatggtgctgaggagtgttaa

SEQ ID NO: 2 - DNA, 30 bp, synthetic construct
  misc_feature 1..30: note = primer
  source 1..30: mol_type = other DNA
tgtgtttcaacaaactttatttatgaacac

SEQ ID NO: 3 - DNA, 34 bp, synthetic construct
  misc_feature 1..34: note = primer
  source 1..34: mol_type = other DNA
ccggaattcaagtagaagattgagatgtgttcac

SEQ ID NO: 4 - DNA, 43 bp, synthetic construct
  misc_feature 1..43: note = primer
  source 1..43: mol_type = other DNA
ggaattccatatgtgtgtttcaacaaactttatttatgaacac

SEQ ID NO: 5 - DNA, 50 bp, synthetic construct
  misc_feature 1..50: note = primer
  source 1..50: mol_type = other DNA
cggcatggacgagctgtacaagtaaaagtagaagattgagatgtgttcac

SEQ ID NO: 6 - DNA, 58 bp, synthetic construct
  misc_feature 1..58: note = primer
  source 1..58: mol_type = other DNA
atcatatgactagtcccgggaattcgagagaaaataattcagaaaaaaataaaattcc

SEQ ID NO: 7 - DNA, 53 bp, synthetic construct
  misc_feature 1..53: note = primer
  source 1..53: mol_type = other DNA
cctcgaggtttaaactacgggatcccctcgctagtggcgggcatgataacaca

SEQ ID NO: 8 - DNA, 43 bp, synthetic construct
  misc_feature 1..43: note = primer
  source 1..43: mol_type = other DNA
cctcgaggtttaaactacgggatccggggtgaggccgccgccg

SEQ ID NO: 9 - DNA, 25 bp, synthetic construct
  misc_feature 1..25: note = primer
  source 1..25: mol_type = other DNA
cccacatgcttcggagaaacgccat

SEQ ID NO: 10 - DNA, 37 bp, synthetic construct
  misc_feature 1..37: note = primer
  source 1..37: mol_type = other DNA
catccaccggagcgatgtcatctgtcttggcaagttg

SEQ ID NO: 11 - DNA, 37 bp, synthetic construct
  misc_feature 1..37: note = primer
  source 1..37: mol_type = other DNA
caacttgccaagacagatgacatcgctccggtggatg

SEQ ID NO: 12 - DNA, 35 bp, synthetic construct
  misc_feature 1..35: note = primer
  source 1..35: mol_type = other DNA
gcctcgaggtttaaactaccatgtcggcactttgc

SEQ ID NO: 13 - DNA, 40 bp, synthetic construct
  misc_feature 1..40: note = primer
  source 1..40: mol_type = other DNA
ggacaacttgccaagacagatgaggcggacgagctcctcc

SEQ ID NO: 14 - DNA, 40 bp, synthetic construct
  misc_feature 1..40: note = primer
  source 1..40: mol_type = other DNA
cgaggtggttttcatctgtcagcaagggcgaggcagtgat

SEQ ID NO: 15 - DNA, 40 bp, synthetic construct
  misc_feature 1..40: note = primer
  source 1..40: mol_type = other DNA
atcactgcctcgcccttgctgacagatgaaaaccacctcg

SEQ ID NO: 16 - DNA, 52 bp, synthetic construct
  misc_feature 1..52: note = primer
  source 1..52: mol_type = other DNA
gctgtggaagtcgactgcgcgaccccttcgattttgtgtatttaccgaggtg

SEQ ID NO: 17 - DNA, 52 bp, synthetic construct
  misc_feature 1..52: note = primer
  source 1..52: mol_type = other DNA
cacctcggtaaatacacaaaatcgaaggggtcgcgcagtcgacttccacagc

SEQ ID NO: 18 - DNA, 35 bp, synthetic construct
  misc_feature 1..35: note = primer
  source 1..35: mol_type = other DNA
ggaattccatatggcgtttctccgaagcatgtggg

SEQ ID NO: 19 - DNA, 34 bp, synthetic construct
  misc_feature 1..34: note = primer
  source 1..34: mol_type = other DNA
cgcggatcctcttacttgtacagctcgtccatgc

SEQ ID NO: 20 - DNA, 55 bp, synthetic construct
  misc_feature 1..55: note = primer
  source 1..55: mol_type = other DNA
gcatggacgagctgtacaagcatcaccatcaccatcactaagaggatccggctgc

SEQ ID NO: 21 - DNA, 55 bp, synthetic construct
  misc_feature 1..55: note = primer
  source 1..55: mol_type = other DNA
gcagccggatcctcttagtgatggtgatggtgatgcttgtacagctcgtccatgc

SEQ ID NO: 22 - DNA, 17 bp, synthetic construct
  misc_feature 1..17: note = primer
  source 1..17: mol_type = other DNA
atggtgggtcggaacag

SEQ ID NO: 23 - DNA, 20 bp, synthetic construct
  misc_feature 1..20: note = primer
  source 1..20: mol_type = other DNA
ttccacatcatcttcagcca

SEQ ID NO: 24 - DNA, 39 bp, synthetic construct
  misc_feature 1..39: note = primer
  source 1..39: mol_type = other DNA
cgaggtttaaactacgggatccatggtgggtcggaacag

SEQ ID NO: 25 - DNA, 45 bp, synthetic construct
  misc_feature 1..45: note = primer
  source 1..45: mol_type = other DNA
ccttgctcacagaaccaccaccaccttccacatcatcttcagcca

SEQ ID NO: 26 - DNA, 40 bp, synthetic construct
  misc_feature 1..40: note = primer
  source 1..40: mol_type = other DNA
cgaggtttaaactacgggatccatgccggaggtgtcatct

SEQ ID NO: 27 - DNA, 45 bp, synthetic construct
  misc_feature 1..45: note = primer
  source 1..45: mol_type = other DNA
ccttgctcacagaaccaccaccacccttggagctggtgtacttag

SEQ ID NO: 28 - RNA, 30 nt, Homo sapiens
  source 1..30: mol_type = mRNA
tcttctttatatacctgccactccgcccta

SEQ ID NO: 29 - RNA, 17 nt, Homo sapiens
  source 1..17: mol_type = rRNA
gccccgcgggacactca

SEQ ID NO: 30 - RNA, 45 nt, synthetic construct
  misc_feature 1..45: note = RNA Adaptor
  source 1..45: mol_type = other RNA
gctgatggcgatgaatgaacactgcgtttgctggctttgatgaaa

SEQ ID NO: 31 - DNA, 20 bp, synthetic construct
  misc_feature 1..20: note = primer
  source 1..20: mol_type = other DNA
gttcattcatcgccatcagc

SEQ ID NO: 32 - DNA, 22 bp, synthetic construct
  misc_feature 1..22: note = primer
  source 1..22: mol_type = other DNA
tttcatcaaagccagcaaacgc

SEQ ID NO: 33 - DNA, 35 bp, synthetic construct
  misc_feature 1..35: note = primer
  source 1..35: mol_type = other DNA
cgcggatccgaacactgcgtttgctggctttgatg

SEQ ID NO: 34 - DNA, 24 bp, synthetic construct
  misc_feature 1..24: note = primer
  source 1..24: mol_type = other DNA
gctgatggcgatgaatgaacactg

SEQ ID NO: 35 - DNA, 20 bp, synthetic construct
  misc_feature 1..20: note = primer
  source 1..20: mol_type = other DNA
ccctccaacgctgggcaatt

SEQ ID NO: 36 - DNA, 21 bp, synthetic construct
  misc_feature 1..21: note = primer
  source 1..21: mol_type = other DNA
ccaagacagatgaaaaccacc

SEQ ID NO: 37 - DNA, 20 bp, synthetic construct
  misc_feature 1..20: note = primer
  source 1..20: mol_type = other DNA
aagccacggtgttctgtgat

SEQ ID NO: 38 - DNA, 20 bp, synthetic construct
  misc_feature 1..20: note = primer
  source 1..20: mol_type = other DNA
gcaggcagaactcatctagg

SEQ ID NO: 39 - DNA, 20 bp, synthetic construct
  misc_feature 1..20: note = primer
  source 1..20: mol_type = other DNA
ggattgcggtttcccttcat

SEQ ID NO: 40 - DNA, 22 bp, Homo sapiens
  source 1..22: mol_type = genomic DNA
gggtataattgtctgggtcgcc

SEQ ID NO: 41 - DNA, 20 bp, Homo sapiens
  source 1..20: mol_type = genomic DNA
agacgccctcggcttacttc

SEQ ID NO: 42 - DNA, 20 bp, Homo sapiens
  source 1..20: mol_type = genomic DNA
agatgtggcgggttttaggg

SEQ ID NO: 43 - DNA, 20 bp, Homo sapiens
  source 1..20: mol_type = genomic DNA
actacaacccttcgctgacg

SEQ ID NO: 44 - DNA, 20 bp, Homo sapiens
  source 1..20: mol_type = genomic DNA
gtgactcccggactagtgga

SEQ ID NO: 45 - DNA, 20 bp, Homo sapiens
  source 1..20: mol_type = genomic DNA
gatgggaagaacacccgagg

SEQ ID NO: 46 - DNA, 20 bp, Homo sapiens
  misc_feature 1..20: note = primer
  source 1..20: mol_type = genomic DNA
caccacagctcttccactca

SEQ ID NO: 47 - DNA, 20 bp, Homo sapiens
  source 1..20: mol_type = genomic DNA
ggggagggatacagtggagt

SEQ ID NO: 48 - protein, 51 aa, Homo sapiens
MAFLRSMWGVLSALGRSGAELCTGCGSRLRSPFSFVYLPRWFSSVLASCPK

SEQ ID NO: 49 - protein, 9 aa, Homo sapiens
SSVLASCPK

SEQ ID NO: 50 - protein, 51 aa, Homo sapiens; identical to SEQ ID NO: 48

SEQ ID NO: 51 - protein, 11 aa, Homo sapiens
SMWGVLSALGR

SEQ ID NO: 52 - protein, 12 aa, Homo sapiens
SGAELCSGCGSR

SEQ ID NO: 53 - protein, 10 aa, Homo sapiens
SPFSFVYLPR

SEQ ID NO: 54 - protein, 11 aa, Homo sapiens
WFSSVLASCPK

SEQ ID NO: 55 - protein, 51 aa, Macaca mulatta
MAFLRSMWGVLNALGRSGAELCSGCGSRLRSPFSFVYLPKWFSSVLASCPK

SEQ ID NO: 56 - protein, 11 aa, Macaca mulatta
SMWGVLNALGR

SEQ ID NO: 57 - protein, 10 aa, Macaca mulatta
SPFSFVYLPK

SEQ ID NO: 58 - protein, 44 aa, Homo sapiens
MAFLRSMWGVLTALGRSGAELCTGCGSRLRSPFSFAYLPRWFSS

SEQ ID NO: 59 - protein, 44 aa, Macaca mulatta
MAFLRSMWGVLNALGRSGAELCSGCGSRLRSPFSFVYLPKWFSS

SEQ ID NO: 60 - protein, 44 aa, Bos taurus
MALLRGVWGVLNALGKSGADLCAGCGSRLRYPFSFAYVPKWFSS

SEQ ID NO: 61 - protein, 44 aa, Oryctolagus cuniculus
MALLRGMWGALRTLGKSGADLCAGCGSRLRSPFSFSYIPRWCSS

SEQ ID NO: 62 - protein, 44 aa, Mus musculus
MALFRGMWSVLKALGRTGVEMCAGCGGRIPSSISLVCIPKCFSS

SEQ ID NO: 63 - protein, 44 aa, Sus scrofa
MALLRGVWGVLSALGKSGADLCAVCGSRLRSPFSFAYVPRWFSS

SEQ ID NO: 64 - protein, 44 aa, Canis lupus
MALFRGVWSVLSALGKSGADLCAGCGSRLRSPFSFAYVPRCFSS

SEQ ID NO: 65 - protein, 44 aa, Galeopterus variegatus
MALFRGVWRVLSSLGKSGAELCAGCGSRLRLPFSFVYIPKWFSS

SEQ ID NO: 66 - protein, 44 aa, Echinops telfairi
MALLRGVWGVLSALGKTGADLCAGCGSRLRFPLSFVCLPRWFSS

SEQ ID NO: 67 - protein, 44 aa, Centella asiatica
MALFRGVWGVLSALGKSGAELCAGCGSRLRFPLRFVYIPRWFSS

SEQ ID NO: 68 - protein, 44 aa, Pipistrellus kuhlii
MPLLRGVWGLLSALGKSGAELCADCGSRLRAPFSFVYIPRWFSS

SEQ ID NO: 69 - RNA, 623 nt, Homo sapiens
  source 1..623: mol_type = mRNA
taaaagtagaagattgagatgtgttcacaatggataggcacaggaaaccagttaggtctcaatacctgaagctatcgtaaaattaagaaaggataaagttggtaaaccttttatatttagtatctttttattcagctcatggacttctgccagcataatacttgctttggaaaacccagataaaggttcatgcaaactttattttgtgtttaggaactactgaggatcagagtaatccaagcaaatgtgaatcattttacctttgacaaaggtaaatcagactatgaagttttttttatacaggatgatgactatggaaagagtactcttgtttccttatattatggaggcaggagtttcgttttcaaaattgttacaaattgtagaagccacggtgttctgtgatataagtgtgtgtttttcataaagcaggcagaactcatctaggtaaattacagttcctaggtataattcacattgtattcagagttgatggttgtacatataagtgattgctggttttagttgcaactttgtataaaagggactgagaaatttataaacttttttcttactgtcttttttctaaagtaaaaacaaagaaattatgtgccaaaaaaaaa

SEQ ID NO: 70 - RNA, 61 nt, Homo sapiens
  source 1..61: mol_type = mRNA
atgaatatttagtgttcataaataaagtttgttgaaacacaaccaagatcattcttttact

SEQ ID NO: 71 - RNA, 102 nt, Homo sapiens
  source 1..102: mol_type = mRNA
  variation 6..32: note = n can be any nucleotide a, c, g, t/u.
tgtatnnnnnnnnnnnnnnnnnnnnnnnnnnttttttcttactgtcttttttctaaagtaaaaacaaagaaattatgtgccagatttatgcatattatttta

SEQ ID NO: 72 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 73 - protein, 12 aa, Homo sapiens
AEWQVYKEEISR

SEQ ID NO: 74 - protein, 13 aa, Homo sapiens
AQNPDAKTTELIR

SEQ ID NO: 75 - protein, 9 aa, Homo sapiens
ELTLLGKPK

SEQ ID NO: 76 - protein, 13 aa, Homo sapiens
ELYIQHAKEDETR

SEQ ID NO: 77 - protein, 13 aa, Homo sapiens
  SITE 9: note = oxidized methionine
EQLTPSQIMSLEK

SEQ ID NO: 78 - protein, 13 aa, Homo sapiens
EQLTPSQIMSLEK

SEQ ID NO: 79 - protein, 11 aa, Homo sapiens
  SITE 11: note = oxidized methionine
FKEQLTPSQIM

SEQ ID NO: 80 - protein, 11 aa, Homo sapiens
FKEQLTPSQIM

SEQ ID NO: 81 - protein, 15 aa, Homo sapiens
  SITE 11: note = oxidized methionine
FKEQLTPSQIMSLEK

SEQ ID NO: 82 - protein, 15 aa, Homo sapiens
FKEQLTPSQIMSLEK

SEQ ID NO: 83 - protein, 10 aa, Homo sapiens
FSKEQLPIFK

SEQ ID NO: 84 - protein, 10 aa, Homo sapiens
FSSVLASCPK

SEQ ID NO: 85 - protein, 7 aa, Homo sapiens
IYQDAYR

SEQ ID NO: 86 - protein, 10 aa, Homo sapiens
KELTLLGKPK

SEQ ID NO: 87 - protein, 8 aa, Homo sapiens
KIYQDAYR

SEQ ID NO: 88 - protein, 14 aa, Homo sapiens
LSDSEKELYIQHAK

SEQ ID NO: 89 - protein, 7 aa, Homo sapiens
LYIQHAK

SEQ ID NO: 90 - protein, 15 aa, Homo sapiens
NLSDSEKELYIQHAK

SEQ ID NO: 91 - protein, 10 aa, Homo sapiens
  SITE 4: note = asparagine deamidation
SAYNVYVAER

SEQ ID NO: 92 - protein, 10 aa, Homo sapiens
SAYNVYVAER

SEQ ID NO: 93 - protein, 13 aa, Homo sapiens
SDSEKELYIQHAK

SEQ ID NO: 94 - protein, 9 aa, Homo sapiens
SSVLASCPK

SEQ ID NO: 95 - protein, 11 aa, Homo sapiens
  SITE 6: note = Methionine oxidation
SWEEQMIEVGR

SEQ ID NO: 96 - protein, 11 aa, Homo sapiens
SWEEQMIEVGR

SEQ ID NO: 97 - protein, 12 aa, Homo sapiens
  SITE 6: note = Methionine oxidation
SWEEQMIEVGRK

SEQ ID NO: 98 - protein, 12 aa, Homo sapiens
SWEEQMIEVGRK

SEQ ID NO: 99 - protein, 8 aa, Homo sapiens
WRELPDSK

SEQ ID NO: 100 - protein, 246 aa, Homo sapiens [sequence omitted]

SEQ ID NO: 101 - protein, 7 aa, Homo sapiens
AEWQVYK

SEQ ID NO: 102 - protein, 12 aa, Homo sapiens
AEWQVYKEEISR

SEQ ID NO: 103 - protein, 7 aa, Homo sapiens
ELTLLGK

SEQ ID NO: 104 - protein, 9 aa, Homo sapiens
ELTLLGKPK

SEQ ID NO: 105 - protein, 8 aa, Homo sapiens
ELYIQHAK

SEQ ID NO: 106 - protein, 7 aa, Homo sapiens
EQLPIFK

SEQ ID NO: 107 - protein, 13 aa, Homo sapiens
  SITE 9: note = Methionine oxidation
EQLTPSQIMSLEK

SEQ ID NO: 108 - protein, 13 aa, Homo sapiens
EQLTPSQIMSLEK

SEQ ID NO: 109 - protein, 15 aa, Homo sapiens
FKEQLTPSQIMSLEK

SEQ ID NO: 110 - protein, 15 aa, Homo sapiens
FKEQLTPSQIMSLEK

SEQ ID NO: 111 - protein, 7 aa, Homo sapiens
IYQDAYR

SEQ ID NO: 112 - protein, 8 aa, Homo sapiens
KIYQDAYR

SEQ ID NO: 113 - protein, 8 aa, Homo sapiens
KPVSSYLR

SEQ ID NO: 114 - protein, 15 aa, Homo sapiens
NLSDSEKELYIQHAK

SEQ ID NO: 115 - protein, 10 aa, Homo sapiens
SAYNVYVAER

SEQ ID NO: 116 - protein, 11 aa, Homo sapiens
  SITE 2: note = Methionine oxidation
SMWGVLTALGR

SEQ ID NO: 117 - protein, 11 aa, Homo sapiens
SMWGVLTALGR

SEQ ID NO: 118 - protein, 10 aa, Homo sapiens
SPFSFVYLPR

SEQ ID NO: 119 - protein, 12 aa, Homo sapiens
SWEEQMIEVGRK

SEQ ID NO: 120 - protein, 6 aa, Homo sapiens
TTELIR

SEQ ID NO: 121 - protein, 11 aa, Homo sapiens
WFSSVLASCPK

SEQ ID NO: 122 - protein, 8 aa, Homo sapiens
WRELPDSK

SEQ ID NO: 123 - protein, 248 aa, synthetic construct
  REGION 1..248: note = primer
MAFLRSMWGVLNALGRSGAELCSGCGSRLRSPFSFVYLPKWFSSVLASCPKKPVSSYLRFSKQQLPIYRAQNPDAKTTELVRRIAKHWRELPDSEKKIYRDAYKAEWEVYKEKISKFKEQLTPDEITSLEKEILDKRLKRKAVTKRKELIQLGKPKRPRSAYNVYVAERFQEAKGESPQEKLKTVKENWKNLSDSEKELYIQHAKEDEIRYHNEMKSWEEHMIEIGRKDVLRRTIKQRQKYGAEEYYK

SEQ ID NO: 124 - protein, 11 aa, Macaca mulatta
DAYKAEWEVYK

SEQ ID NO: 125 - protein, 8 aa, Macaca mulatta
ELYIQHAK

SEQ ID NO: 126 - protein, 13 aa, Macaca mulatta
ELYIQHAKEDEIR

SEQ ID NO: 127 - protein, 13 aa, Macaca mulatta
EQLTPDEITSLEK

SEQ ID NO: 128 - protein, 15 aa, Macaca mulatta
FKEQLTPDEITSLEK

SEQ ID NO: 129 - protein, 12 aa, Macaca mulatta
FQEAKGESPQEK

SEQ ID NO: 130 - protein, 12 aa, Macaca mulatta
LRSPFSFVYLPK

SEQ ID NO: 131 - protein, 15 aa, Macaca mulatta
NLSDSEKELYIQHAK

SEQ ID NO: 132 - protein, 20 aa, Macaca mulatta
NLSDSEKELYIQHAKEDEIR

SEQ ID NO: 133 - protein, 10 aa, Macaca mulatta
QKYGAEEYYK

SEQ ID NO: 134 - protein, 7 aa, Macaca mulatta
QQLPYIR

SEQ ID NO: 135 - protein, 10 aa, Macaca mulatta
SAYNVYVAER

SEQ ID NO: 136 - protein, 12 aa, Macaca mulatta
SGAELCSGCGSR

SEQ ID NO: 137 - protein, 11 aa, Macaca mulatta
  SITE 2: note = Methionine oxidation
SMWGVLNALGR

SEQ ID NO: 138 - protein, 10 aa, Macaca mulatta
SPFSFVYLPK

SEQ ID NO: 139 - protein, 11 aa, Macaca mulatta
  SITE 6: note = Methionine oxidation
SWEEHMIEIGR

SEQ ID NO: 140 - protein, 11 aa, Macaca mulatta
SWEEHMIEIGR

SEQ ID NO: 141 - protein, 12 aa, Macaca mulatta
  SITE 6: note = Methionine oxidation
SWEEHMIEIGRK

SEQ ID NO: 142 - protein, 12 aa, Macaca mulatta
SWEEHMIEIGRK

SEQ ID NO: 143 - protein, 11 aa, Macaca mulatta
WFSSVLASCPK

SEQ ID NO: 144 - protein, 8 aa, Macaca mulatta
YGAEEYYK